(19) United States
(12) Patent Application Publication
(10) Pub. No.: US 2023/0310440 A1
(43) Pub. Date: Oct. 5, 2023

(54) RESPIRATORY STIMULANT NASAL FORMULATIONS

(71) Applicant: Enalare Therapeutics Inc., Princeton, NJ (US)

(72) Inventors: Frank Diana, Princeton, NJ (US); Joseph V. Pergolizzi, Princeton, NJ (US)

(21) Appl. No.: 18/129,538

(22) Filed: Mar. 31, 2023

Related U.S. Application Data
(60) Provisional application No. 63/326,578, filed on Apr. 1, 2022.

(51) Int. Cl. A61K 31/53; A61K 9/00; A61K 47/10; A61K 47/40; A61K 47/12

(52) U.S. Cl. CPC A61K 31/53; A61K 9/0043; A61K 47/10; A61K 47/40; A61K 47/12

(57) ABSTRACT

Disclosed in certain embodiments a nasal formulation comprising a compound of Formula (I) as disclosed herein and a pharmaceutically acceptable excipient.

### FIELD OF THE DISCLOSURE

The present disclosure relates methods and compositions to treat respiratory depression, e.g., modulated by an opioid or a non-opioid agent, inflammation or infection.

### BACKGROUND OF THE DISCLOSURE

The human body is critically dependent on the ventilatory control system for adequate uptake of oxygen and removal of carbon dioxide (CO₂). Many active agents such as opioid analgesics, through their actions on μ-opioid receptor expressed on respiratory neurons in the brainstem, may cause respiratory depression in certain situations such as overdose.

Other agents may cause respiratory depression in overdose or other situations. For example, anesthetics such as propofol can cause respiratory depression that may be life threatening.

Respiratory depression may also be caused by non-pharmacological reasons such as inflammation or infection.

There exists a need in the art for methods of treatment and formulations to treat respiratory depression caused by any reason.

### SUMMARY OF THE DISCLOSURE

In certain embodiments, the present disclosure is directed to methods of nasal treatment and formulations to treat respiratory depression caused by, e.g., opioid agents, non-opioid agents, inflammation or infection.

In certain embodiments, the present disclosure is directed to a nasal formulation comprising from about 0.01% w/w to about 10% w/w of a compound of Formula (I) and a pharmaceutically acceptable excipient.

In certain embodiments, the present disclosure is directed to a nasal formulation comprising a compound of Formula (I) as disclosed herein and a pharmaceutically acceptable excipient, wherein the formulation maintains at least 90% of the compound after accelerated storage conditions of 25° C. at 60% relative humidity for 2 weeks.

In certain embodiments, the present invention is directed to nasal compositions and methods for: (1) the treatment of patients who have been subjected to drug overdose, whether by accidental or other means; (2) the treatment of patients who have been subjected to opioid overdose, whether by accidental or other means; (3) the treatment of patients who have opioid withdrawal symptoms, whether by accidental or other means, or (4) the prophylactic treatment of patients for opioid withdrawal symptoms, whether by accidental or other means;

In certain embodiments, the nasal formulation can be contained in a nasal delivery device such as a metered dose device, dropper, spray, pump spray, nebulizer, atomizer, squeeze tube, squeeze bottle, pipette, ampule, nasal cannula, nasal spray inhaler, nasal continuous positive air pressure device, or breath actuated bi-directional delivery device.

### Definitions

As used herein, the singular forms “a,” “an,” and “the” include plural references unless the context clearly indicates otherwise. Thus, for example, reference to “an active agent” includes a single active agent as well as a mixture of two or more different active agent, and reference to an “excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

As used herein, the terms “active agent,” “active ingredient,” and “active pharmaceutical ingredient” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is nonsuperimposable on its mirror image and hence optically active wherein the enantiomer rotates the plane of polarized light in one direction by a certain degree, and its mirror image rotates the plane of polarized light by the same degree but in the opposite direction.

The term “chiral center” refers to an atom (e.g., a carbon atom) to which four different groups are attached.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

“Pharmaceutically acceptable salts” or “salts” include, but are not limited to, inorganic acid salts such as hydrochloride, hydrobromide, hydroiodic, sulfate, hydrogen sulfate, phosphate, nitric, carbonic, sulfuric, phosphoric (including hydrogen phosphate and dihydrogen phosphate), and the like; organic acid salts such as an oxalate, a malonate, a citrate, a fumarate, a lactate, a malate, a succinate, formate, acetate, trifluoroacetate, maleate, tartrate, a gluconate, a benzoate, a salicylate, a xinafoate, a pamoate, an ascorbate, an adipate, a cinnamate, and the like; sulfonates such as methanesulfonate, benzenesulfonate, p-toluenesulfonate and the like; amino acid salts such as arginate, asparaginate, glutamate and the like; metal salts such as zinc salt, sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt, chloroprocaine, choline, diethanolamine, ethylenediamine, meglumine (N-methylglucamine) and procaine, and the like. These salts may be present in the form of a hydrate, a solvate, or a crystalline polymorph. In certain embodiments, appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. All of these salts may be prepared by conventional means from the corresponding compound of the invention by reacting, for example, the appropriate acid or base with the compound of the invention. Handbook of Pharmaceutical Salts: Properties, and Use (P. H. Stahl & C. G. Wermuth eds., Verlag Helvetica Chimica Acta, 2002) [1].

The term “disease” or “diseases” or “condition” or “conditions” refers to those medical conditions that can be treated or prevented by administration to a subject of an effective amount of an active agent.

The terms “treatment of” and “treating” includes the lessening of the severity of or cessation of a condition or lessening the severity of or cessation of symptoms of a condition. In certain embodiments, the terms “treatment” or “treating” with respect to a condition means administration with the intent to provide a pharmacodynamics effect, regardless of the outcome. In certain embodiments, “treatment” or “treating” means “having positive effect on a condition” and encompass reduction in the severity, amelioration, and/or alleviation of at least one symptom of a condition; a reduction, amelioration, and/or alleviation in the severity of the conditions; delay, prevention, or inhibition of the progression of the condition; or a perceived improvement or benefit as a result of the treatment. Treatment, as used herein, does not require total curing of the condition. In certain embodiments, a composition of the present disclosure may provide improvement to a patient's quality of life, or delay, prevent, inhibit the onset of one or more symptoms of a condition, or provide a perceived benefit.

The terms “prevention of” and “preventing” includes the avoidance of the onset of a condition.

The term “therapeutically effective amount” is intended to include an amount of an active agent, or an amount of the combination of active agents, e.g., to treat or prevent the condition, or to treat the symptoms of the condition, in a subject.

The term “effective amount” is intended to include an amount of a component, or an amount of a combination of component, to achieve a certain result or property, for instance, an effective amount of a pH adjusting agent to achieve a pH of 6.0 is intended to include an amount of one or more pH adjusting agents to arrive at a pH of 6.0.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

As used herein, the term “alkyl,” by itself or as part of another substituent means, unless otherwise stated, a straight or branched chain hydrocarbon having the number of carbon atoms designated (i.e. C1-C10 means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C1-C6)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e. C3-C6 means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C3-C6)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH2-CH═CH2.

As used herein, the term “alkynyl,” employed alone or in combination with other terms, means, unless otherwise stated, a stable straight chain or branched chain hydrocarbon group with a triple carbon-carbon bond, having the stated number of carbon atoms. Examples include ethynyl and propynyl, and the higher homologs and isomers.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH2, —N(CH3)2, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH2, —C(═O)NH(C1-C4)alkyl, —C(═O)N((C1-C4)alkyl)2, —SO2NH2, —C(═NH)NH2, and —NO2, preferably containing one or two substituents selected from halogen, —OH, alkoxy, —NH2, trifluoromethyl, —N(CH3)2, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C1-C3)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH CH₂—OH, —CH₂—CH═N—OCH₃, —CH═CH—N(CH₃) CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### BRIEF DESCRIPTION OF THE DRAWINGS

FIG. 1 is a graph showing the pH stability profiles at 60° C. (% impurities) of an Example according to the present disclosure;

FIG. 2. is a graph showing the pH degradation at various temperatures of an Example according to the present disclosure.

### DETAILED DESCRIPTION

In certain embodiments, the present invention is directed to a nasal formulation comprising from about 0.01% w/w to about 10% w/w of a compound of Formula (I) and a pharmaceutically acceptable excipient.

In certain embodiments, the pharmaceutically acceptable excipient comprises a liquid.

In certain embodiments, the compound of Formula (I) is dissolved in the liquid.

In certain embodiments, the compound of Formula (I) is suspended in the liquid.

In certain embodiments, the pharmaceutically acceptable excipient is a collection of particles.

In certain embodiments, the compound of Formula (I) is dispersed within each particle.

In certain embodiments, the compound of Formula (I) is coated on each particle.

In certain embodiments, the pharmaceutically acceptable excipient is a cream, ointment or gel.

In certain embodiments, the nasal formulation of maintains at least 90% of the compound after accelerated storage conditions of 40° C. at 75% relative humidity for 2 weeks.

In certain embodiments, the pH of the formulation is from about 3.5 to about 5.5.

In certain embodiments, the pH is from about 4 to about 5.

In certain embodiments, the pH is selected from about 4.0, about 4.5 or about 5.0.

In certain embodiments, the concentration of the compound is from about 1 mg/mL to about 100 mg/mL.

In certain embodiments, the excipient is selected from water, ethanol, polyalkylene glycol, alkylene glycol, cyclodextrin, saline, ringers solution, dextrose, polyethylene glycol-hydroxystearate or a combination thereof.

In certain embodiments, the excipient comprises ethanol.

In certain embodiments, the excipient comprises ethanol in an amount of from about 1% to about 30%, about 5% to about 25% or about 10% to about 20%.

In certain embodiments, the excipient comprises propylene glycol.

In certain embodiments, the excipient comprises propylene glycol in an amount of from about 20% to about 100%, about 50% to about 95% or about 70% to about 90%.

In certain embodiments, the excipient comprises polyethylene glycol.

In certain embodiments, the excipient comprises polyethylene glycol in an amount of from about 20% to about 100%, about 50% to about 95% or about 70% to about 90%.

In certain embodiments, the excipient comprises hydroxypropyl-p-cyclodextrin.

In certain embodiments, the excipient comprises hydroxypropyl-p-cyclodextrin in an amount of from about 1% to about 50%, about 10% to about 40% or about 20% to about 30%.

In certain embodiments, the nasal formulation comprises a buffer solution.

In certain embodiments, the buffer solution comprises a citric acid, glycine, citrate or acetate.

In certain embodiments, the buffer solution comprise a citrate.

In certain embodiments, the buffer solution comprise an acetate.

In certain embodiments, the present invention is directed to a method of providing respiratory stimulation comprising administering intranasally a nasal formulation as disclosed herein.

In certain embodiments, the present invention is directed to a method of treating respiratory depression comprising nasally administering a formulation as disclosed herein.

In certain embodiments, the respiratory depression is caused by an opioid agent.

In certain embodiments, the respiratory depression is caused by a non-opioid agent.

In certain embodiments, the respiratory depression is caused by inflammation.

In certain embodiments, the respiratory depression is caused by infection.

Certain embodiments of the instant disclosure are directed to a nasal formulation comprising a compound of Formula (I) and a pharmaceutically acceptable excipient, wherein the compound of Formula (I) is selected from:

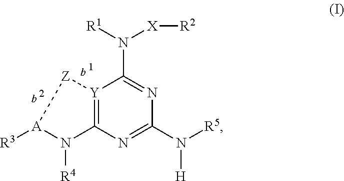

wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl, substituted alkynyl, cycloalkyl, substituted cycloalkyl, alkenyl, substituted alkenyl, —NR¹R², —C(O)OR¹, acyl, or aryl;
R⁴ is H, alkyl, or substituted alkyl;
R⁵ is H, alkyl, propargylic, substituted propargylic, homopropargylic, substituted homopropargylic, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C; or a salt thereof.

Certain embodiments of the instant disclosure are directed to a nasal formulation comprising a compound of Formula (I) and a pharmaceutically acceptable excipient, wherein the compound of Formula (I) is selected from:

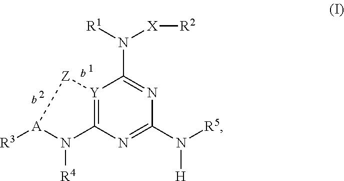

wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl, substituted alkynyl, cycloalkyl, substituted cycloalkyl, alkenyl, substituted alkenyl, —NR¹R², —C(O)OR¹, acyl, or aryl;
R⁴ is H, alkyl, or substituted alkyl;
R⁵ is H, alkyl, propargylic, substituted propargylic, homopropargylic, substituted homopropargylic, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl; wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b^{W} is a single bond, and: (i) Z is CH₂, bond b² is a single bond, and A is CH; or, (ii) Z is CH, bond b² is a double bond, and A is C; or a salt thereof.

Certain embodiments of the instant disclosure are directed to a nasal formulation comprising a compound of Formula (I) and a pharmaceutically acceptable excipient, wherein the compound of Formula (I) is selected from:

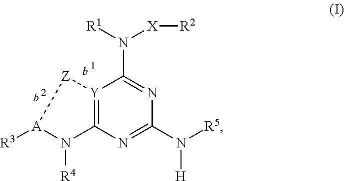

wherein:
R¹ and R² are independently H, alkyl, substituted alkyl, cycloalkyl, substituted cycloalkyl, alkenyl, substituted alkenyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, cycloalkyl, substituted cycloalkyl, alkenyl, substituted alkenyl, —NR¹R², —C(O)OR¹, acyl, or aryl;
R⁴ is H, alkyl, or substituted alkyl;
R⁵ is H, alkyl, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C; or a salt thereof.

In one embodiment, R³ is H, alkyl, substituted alkyl, cycloalkyl, substituted cycloalkyl, alkenyl, or substituted alkenyl. In another embodiment, R⁵ is H, alkyl, substituted alkyl, cycloalkyl, substituted cycloalkyl, alkenyl, substituted alkenyl, or acyl.

Certain embodiments of the instant disclosure are directed to a nasal formulation comprising a compound of Formula (I) and a pharmaceutically acceptable excipient, wherein the compound of Formula (I) is selected from:

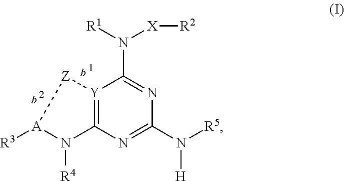

R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl or substituted alkynyl;
R⁴ is H, alkyl, or substituted alkyl;
R⁵ is alkyl, propargylic, substituted propargylic, homopropargylic, or substituted homopropargylic, wherein at least one substituent selected from the group consisting of R¹, R², R³ and R⁵ is alkynyl or substituted alkynyl;
R⁶ is H, alkyl, substituted alkyl or alkenyl;
X is a bond, O or NR⁴; and,
Y is N, CR⁶ or C; wherein:
if Y is N or CR⁶, then bond b¹ is nil and:
   (i) Z is H, bond b² is a single bond, and A is CH; or,
   (ii) Z is nil, bond b² is nil, and A is a single bond; and,
if Y is C, then bond b^{W} is a single bond, and:
   (i) Z is CH₂, bond b² is a single bond, and A is CH; or,
   (ii) Z is CH, bond b² is a double bond, and A is C;
      or a salt thereof.

In certain embodiments, (i) R³ is H, alkyl or substituted alkyl, and R⁵ is propargylic, substituted propargylic, homopropargylic, or substituted homopropargylic, or (ii) R³ is H or alkynyl, and R⁵ is alkyl, propargylic, substituted propargylic, homopropargylic, or substituted homopropargylic.

In one embodiment, the at least one compound of formula (I) is selected from the group consisting of: (i) Y is N, bond b1 is nil, Z is H, bond b2 is a single bond, A is CH, and the at least one compound is a compound of formula (II-a) or a salt thereof:

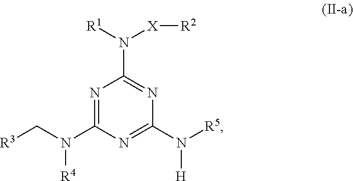

and
(ii) Y is N, bond b1 is nil, Z is nil, bond b2 is nil, and A is a bond, and the compound of the invention is a compound of formula (II-b) or a salt thereof:

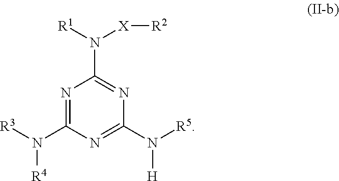

In one embodiment, the at least one compound of formula (I) is selected from the group consisting of: (i) Y is CR⁶, bond b¹ is nil, Z is H, bond b² is a single bond, A is CH, and the at least one compound is a compound of formula (III-a) or a salt thereof:

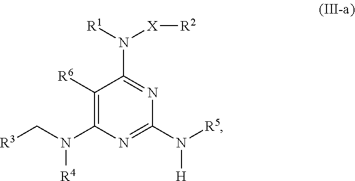

and
(ii) Y is CR⁶, bond b is nil, Z is nil, bond b² is nil, and A is a bond, and the compound of the invention is a pyrimidine of formula (III-b) or a salt thereof:

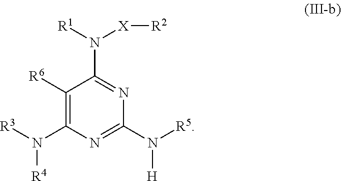

In one embodiment, Y is C, bond b is a single bond, Z is CH₂, bond b² is a single bond, A is CH, and said at least one compound is a compound of formula (IV) or a salt thereof:

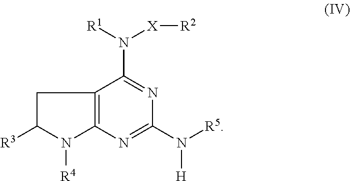

In one embodiment, Y is C, bond b is a single bond, Z is CH, bond b² is a double bond, A is C, and said at least one compound is a compound of formula (V) or a salt thereof:

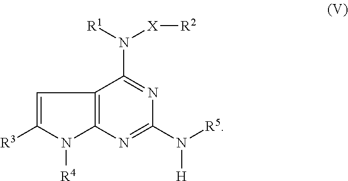

In one embodiment, the at least one compound is selected from the group consisting of: N-(4,6-Bis-methylamino-[1,3,5]triazin-2-yl)-N,O-dimethyl-hydroxylamine (XX), N-(4,6-Bis-ethylamino-[1,3,5]triazin-2-yl)-N,O-dimethyl-hydroxylamine (XXII), N-(4-Cyclopropylmethylamino)-N-(6-n-propylamino) [1,3,5]triazin-2-yl)-N,O-dimethyl-hydroxylamine (XXV), N-(4-Ethylamino)-N-(6-n-propylamino)-[1,3,5]triazin-2-yl)-N,O-dimethyl-hydroxylamine (XXVII), N-(Bis-4,6-(2-methylpropylamino)) [1,3,5]triazin-2-yl)-N,O-dimethyl-hydroxylamine (XXIX), N-(Bis-4,6-(2,2-dimethylpropylamino)) [1,3,5]triazin-2-yl)-O,N-dimethyl-hydroxylamine (XXXI), 4,6-Bis-N-cyclopropylamino-[1,3,5]triazin-2-yl)-N,O-dimethyl-hydroxylamine hydrochloride (XXXIII), N-(4,6-Bis-n-propylamino-[1,3,5]triazin-2-yl)-O,N-dimethyl-hydroxylamine (XXXV), N-(4-(Methoxy(methyl)amino)-6-(propylamino)-1,3,5-triazin-2-yl)propionamide (XL), N-(4,6-Bis-propylamino-[1,3,5]triazin-2-yl)-O-methyl-hydroxylamine (XLI), 0-Allyl-N-(4,6-bis-propylamino-[1,3,5]triazin-2-yl)-hydroxylamine (XLIII), N-(4,6-Bis-propylamino-[1,3,5]triazin-2-yl)-hydroxylamine (XLV), 6-(Methoxy(methyl)amino)-N2-propyl-1,3,5-triazine-2,4-diamine (XLVII), N-(4,6-Bis-propylamino-[1,3,5]triazin-2-yl)-N-methyl-hydroxylamine (XLVIII), O-Benzyl-N-(4,6-bis-propylamino-[1,3,5]triazin-2-yl)-N-methyl-hydroxylamine (LIII), N-(4,6-Bis-propylamino-[1,3,5]triazin-2-yl)-N-isopropyl-hydroxylamine (LV), 6-[1,2]Oxazinan-2-yl-N,N′-dipropyl-[1,3,5]triazine-2,4-diamine (LVII), N-(4,6-Bis-propylamino-[1,3,5]triazin-2-yl)-O-isopropyl-N-methyl-hydroxylamine (LXIV), O-Benzyl-N-(4,6-bis-propylamino-[1,3,5]triazin-2-yl)-N-ethyl-hydroxylamine (LXVIII), N-(4,6-Bis-propylamino-[1,3,5]triazin-2-yl)-O-isopropyl-hydroxylamine (LXX), 6-((Benzyloxy)(isopropyl)amino)-N2,N4-dipropyl-1,3,5-triazine-2,4-diamine (LXXII), N-(4,6-Bis-propylamino-[1,3,5]triazin-2-yl)-N-ethyl-O-isopropyl-hydroxylamine (LXXVI), N-(4,6-Bis-propylamino-[1,3,5]triazin-2-yl)-O-isobutyl-N-methyl-hydroxylamine (LXXXII), 6-(Methyl(thiophen-2-ylmethoxy)amino)-N2,N4-dipropyl-1,3,5-triazine-2,4-diamine (LXXXIV), N-(4,6-Bis-propylamino-[1,3,5]triazin-2-yl)-O-cyclopropylmethyl-N-methyl-hydroxylamine (XCI), N-(4,6-Bis-propylamino-[1,3,5]triazin-2-yl)-O-ethyl-N-methyl-hydroxylamine (XCVI), N-(4,6-Bis-propylamino-[1,3,5]triazin-2-yl)-O-(2,2-difluoro-ethyl)-hydroxylamine (C), 4-N-(2-Dimethylaminoethyl)amino-6-N-(n-propyl)amino-[1,3,5]triazin-2-yl)-N,O-dimethyl-hydroxylamine (CIII), 4-N-(3-(1-N-Methylimidazol-2-yl)-propyl)-amino-6-N-(n-propyl)amino-[1,3,5]triazin-2-yl)-N,O-dimethyl-hydroxylamine (CV), 4-N-(1-N-Methylimidazol-2-yl)-methylamino-6-N-(n-propyl)amino-[1,3,5]triazin-2-yl)-O,N-dimethyl-hydroxylamine (CVII), 4,6-Bis-(N-(2-dimethylaminoethyl)amino)-[1,3,5]triazin-2-yl)-N,O-dimethyl-hydroxylamine (CIX), 4,6-Bis-(N-(pyridin-4-ylmethyl)amino)-[1,3,5]triazin-2-yl)-N,O-dimethyl-hydroxylamine (CXI), 4,6-Bis-[N-(3-methoxy-n-propyl)amino]-[1,3,5]triazin-2-yl)-N,O-dimethyl-hydroxylamine (CXIII), 4,6-Bis-[N-(tetrahydropyran-4-ylmethyl)amino]-[1,3,5]triazin-2-yl)-N,O-dimethyl-hydroxylamine (CXV), N-(5,8,11-Trioxa-2,14,16,18,19-pentaazabicyclo[13.3.1]-nonadeca-1(18),15(19),16(17)-trien-17-yl)-N,O-dimethylhydroxylamine (CXVII), N-(4,6-Bis-propylamino-[1,3,5]triazin-2-yl)-N′,N′-dimethylhydrazine (XLVI), N-(4,6-Bis-propylamino-[1,3,5]triazin-2-yl)-N-methyl-N′-methylhydrazine (XLIX), a salt thereof and mixtures thereof. In another embodiment, the salt is hydrogen sulfate or hydrochloride.

In one embodiment, the at least one compound is 2,6-bis-(N-n-propylamino)-[1,3]pyrimidin-4-yl)-N,O-dimethyl-hydroxylamine N-(4-(Methoxy(methyl)amino)-6-(propylamino)-1,3,5-triazin-2-yl)propionamide or a salt thereof. In another embodiment, the salt is hydrogen sulfate or hydrochloride.

In one embodiment, the at least one compound is N-(4-(Methoxy(methyl)amino)-6-(propylamino)-1,3,5-triazin-2-yl)propionamide or a salt thereof. In another embodiment, the salt is hydrogen sulfate or hydrochloride.

In one embodiment, the at least one compound is selected from the group consisting of: 2-(n-Propyl)amino-4-(i-propylamino-7-methyl-pyrrolidino[2,3-d]pyrimidine (CXXVI), 2-(n-Propyl)amino-4-dimethylamino-7-methyl-pyrrolidino[2,3-d]pyrimidine (CXXVIII), 2-(n-Propyl)amino-4-methylamino-7-methyl-pyrrolidino[2,3-d]pyrimidine (CXXXI), 2-(n-Propyl)amino-4-(i-propyl)amino-7-i-propyl-pyrrolidino[2,3-d]pyrimidine (CXXXVI), 2,4-Bis-(n-propyl)amino-7H-pyrrolidino[2,3-d]pyrimidine (CXLIX), 2-(n-Propyl)amino-4-(4-hydroxypiperidin-1-yl)-7-methyl-pyrrolidino[2,3-d]pyrimidine (CLII), 8-(7-Methyl-2-(propylamino)-pyrrolidino[2,3-d]pyrimidin-4-yl)-8-azabicyclo[3.2.1]octan-3-ol (CLV), a salt thereof and mixtures thereof. In another embodiment, the salt is hydrogen sulfate or hydrochloride.

In one embodiment, the at least one compound is selected from the group consisting of: N-(2-Propylamino-7H-pyrrolo[2,3d]pyrimidin-4-yl)-O,N-dimethyl-hydroxylamine (CXLI), N-(2-(Propen-2-yl)amino-7-methyl-pyrrolo[2,3d]pyrimidin-4-yl)-N,O-dimethyl-hydroxylamine (CLVIII), N-(2-(Propen-2-yl)amino-7-methyl-pyrrolo[2,3d]pyrimidin-4-yl)-O-methyl-hydroxylamine (CLX), N-(2-n-Propylamino-7-methyl-pyrrolo[2,3d]pyrimidin-4-yl)-O,N-dimethyl-hydroxylamine (CLXII), N-(2-n-Propylamino-7-methyl-pyrrolo[2,3d]pyrimidin-4-yl)-O-methyl-hydroxylamine (CLXIV), N-(2-n-Propylamino-7-methyl-pyrrolo[2,3d]pyrimidin-4-yl)-hydrazine (CLXVI), N-Methyl-N-(2-n-propylamino-7-methyl-pyrrolo[2,3d]pyrimidin-4-yl)-hydrazine (CLXVIII), N,N-dimethyl-N′-(2-n-propylamino-7-methyl-pyrrolo[2,3d]pyrimidin-4-yl)-hydrazine (CLXX), a salt thereof and mixtures thereof. In another embodiment, the salt is hydrogen sulfate or hydrochloride.

In certain embodiments, the compound is selected from the group consisting of O,N-dimethyl-N-[4-(n-propylamino)-6-(prop-2-ynylamino-[1,3,5]triazin-2-yl]-hydroxylamine; N-methyl-N′-n-propyl-N″-prop-2-ynyl-[1,3,5]triazine-2,4,6-triamine; a salt thereof; and any combinations thereof.

In certain embodiments the Compound A below is utilized in the present invention is

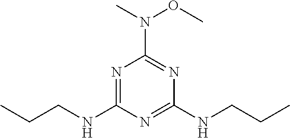 [SMILES: CCCNc1nc(NCCC)nc(N(C)OC)n1]

or a pharmaceutically acceptable salt thereof.

In certain embodiments, the compound of Formula (I) is selected from compounds described in U.S. Pat. No. 9,162,992 and/or in U.S. Pat. No. 9,351,972 and/or in United States Patent Application Publication No. 2015-0291597, now abandoned, the teachings of which are incorporated by reference herein in their entirety.

In certain embodiments, the formulation maintains at least 90% of the compound after accelerated storage conditions of 25° C. at 60% relative humidity for 1 month.

In certain embodiments, the formulation maintains at least 90% of the compound after accelerated storage conditions of 25° C. at 60% relative humidity for 2 months.

In certain embodiments, the formulation maintains at least 90% of the compound after accelerated storage conditions of 25° C. at 60% relative humidity for 3 months.

In certain embodiments, the formulation maintains at least 95% of the compound after accelerated storage conditions of 25° C. at 60% relative humidity for 2 weeks.

In certain embodiments, the formulation maintains at least 95% of the compound after accelerated storage conditions of 25° C. at 60% relative humidity for 1 month.

In certain embodiments, the formulation maintains at least 90% of the compound after accelerated storage conditions of 25° C. at 60% relative humidity for 2 months.

In certain embodiments, the formulation maintains at least 90% of the compound after accelerated storage conditions of 25° C. at 60% relative humidity for 3 months.

In certain embodiments, the formulation maintains at least 90% of the compound after accelerated storage conditions of 40° C. at 75% relative humidity for 2 weeks.

In certain embodiments, the formulation maintains at least 90% of the compound after accelerated storage conditions of 40° C. at 75% relative humidity for 1 month.

In certain embodiments, the formulation maintains at least 90% of the compound after accelerated storage conditions of 40° C. at 75% relative humidity for 2 months.

In certain embodiments, the formulation maintains at least 90% of the compound after accelerated storage conditions of 40° C. at 75% relative humidity for 3 months.

In certain embodiments, the formulation maintains at least 95% of the compound after accelerated storage conditions of 40° C. at 75% relative humidity for 2 weeks.

In certain embodiments, the formulation maintains at least 95% of the compound after accelerated storage conditions of 40° C. at 75% relative humidity for 1 month.

In certain embodiments, the formulation maintains at least 90% of the compound after accelerated storage conditions of 40° C. at 75% relative humidity for 2 months.

In certain embodiments, the formulation maintains at least 90% of the compound after accelerated storage conditions of 40° C. at 75% relative humidity for 3 months.

In certain embodiments, the pH of the formulation is from about 2.5 to about 5.5.

In certain embodiments, the pH of the formulation is from about 3.5 to about 5.5

In certain embodiments, the pH is from about 4 to about 5.

In certain embodiments, the pH is selected from about 4.0, about 4.5 or about 5.0.

In certain embodiments, the concentration of the compound is from about 1 mg/mL to about 50 mg/mL.

In certain embodiments, the concentration of the compound is from about 10 mg/mL to about 30 mg/mL.

In certain embodiments, the concentration of the compound is from about 15 mg/mL to about 25 mg/mL.

In certain embodiments, the concentration of the compound is selected from about 15 mg/mL, about 20 mg/mL or about 25 mg/mL.

In certain embodiments, the excipient is selected from ethanol, polyalkylene glycol, alkylene glycol, cyclodextrin, saline, ringers solution, dextrose or a combination thereof. In certain embodiment, the excipient is polyethylene glycol-hydroxystearate, e.g., obtained by reacting 15 moles of ethylene glycol with 1 mole of 12-hydroxy stearic acid. This is commercially available as Kolliphor® HS15 from BASF.

In certain embodiments, the excipient comprises ethanol.

In certain embodiments, the excipient comprises ethanol in an amount of from about 1% to about 30%, about 5% to about 25% or about 10% to about 20%.

In certain embodiments, the excipient comprises propylene glycol.

In certain embodiments, the excipient comprises propylene glycol in an amount of from about 20% to about 100%, about 50% to about 95% or about 70% to about 90%.

In certain embodiments, the excipient comprises polyethylene glycol.

In certain embodiments, the excipient comprises polyethylene glycol in an amount of from about 20% to about 100%, about 50% to about 95% or about 70% to about 90%.

In certain embodiments, the excipient comprises hydroxypropyl-p-cyclodextrin.

In certain embodiments, the excipient comprises hydroxypropyl-p-cyclodextrin in an amount of from about 1% to about 50%, about 10% to about 40% or about 20% to about 30%.

In certain embodiments, the present invention is directed to a method of providing respiratory stimulation comprising administering intranasally a nasal formulation as disclosed herein.

### Composition

In certain embodiments, the nasal pharmaceutical composition is pre-mixed (e.g., an active agent is pre-mixed with one or more pharmaceutically acceptable excipients and optionally with one or more additional active agents).

In certain embodiments, the pharmaceutical composition may be contained in a glass container or device or in a plastic container or device suitable for intranasal delivery.

In certain embodiments, pharmaceutically acceptable excipients for nasal administration include a pharmaceutically acceptable carrier, such as, a liquid or solid filler, stabilizer, dispersing agent, suspending agent, diluent, thickening agent, solvent or encapsulating material, depending on if the formulation is a liquid or solid. Each carrier should be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the nasal compositions may be brought about by including in the composition an agent that delays absorption, for example, aluminum monostearate or gelatin.

The pharmaceutical preparations may be sterilized and if desired mixed with auxiliary agents, e.g., lubricants, preservatives, stabilizers, wetting agents, emulsifiers, salts for influencing osmotic pressure buffers, coloring, and the like.

Examples of preservatives useful in accordance with the invention included but are not limited to those selected from the group consisting of benzyl alcohol, sorbic acid, parabens, imidurea and combinations thereof.

The composition preferably includes an antioxidant and a chelating agent which inhibit the degradation of the compound. Preferred antioxidants for some compounds are butylated hydroxytoluene (BHT), butylated hydroxyanisole (BHA), alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered or particulate formulations may also be prepared for nasal delivery. Additional excipients, such as fillers and coloring agents, may also be included in these formulations.

A nasal pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate.

In certain embodiments, the one or more additional excipients includes a pH adjusting agent, which may be selected from sodium hydroxide, potassium hydroxide, calcium hydroxide, ammonium hydroxide, sulfuric acid, phosphoric acid, nitric acid, sodium citrate, sodium acetate, magnesium hydroxide, citric acid, hydrochloric acid, or a mixture thereof.

In certain embodiments, the composition may include one or more additional excipients, such as, without limitations, carbohydrates, antioxidants, chelating agents, low-molecular weight proteins, high-molecular weight polymers, gel-forming agents, stabilizers, additives, wetting agents, emulsifying agents, surfactant and/or dispersing agents, alkalizing agents, coloring agents, synthetic dies, fillers, diluents, mineral oxides, preservatives, or a mixture thereof.

In certain embodiment, the composition further includes an antioxidant. In certain embodiments, the antioxidant may include trivalent phosphorous like e.g phosphite, phenolic antioxidants, hydroxylamines, lactones such as substituted benzofuranones. Hindered phenols, thiosynergists and/or hindered amines are useful for the long-term stability for polymers, whereas the following antioxidants are suitable for use also in situation where the active substance is subject to oxidation: acids (ascorbic acid, erythorbic acid, etidronic acid, gallic acid, hypophosphorous acid, nordihydroguairetic acid, propionic acid etc.), phenols (e.g. BHA, BHT, t-butyl hydroquinone, dodecyl gallate, octyl gallate, 1,3,5-trihydroxybenzene), organic and inorganic salts (calcium ascorbate, sodium ascorbate, sodium bisulphite, sodium metabisulfite, sodium sulfite, potassium bisulphite, potassium metabisulphite), esters (calcium ascorbate, dilauryl thiodipropionate, dimyristyl thiodipropionate, distearyl thiodipropionate), pyranon (maltol), and vitamin E (tocopherol, D-α-tocopherol, DL-α-tocopherol, tocopherol acetate, d-α-tocopheryl acetate, dl-α-tocopheryl acetate. However, other anti-oxidative agents known in the art may be used according to the present invention.

In certain embodiments, suitable antioxidants may include, without limitations, sterically hindered phenols, aryl amines, thioureas, thiocarbamates, phosphites, thioether esters, and combinations of the foregoing. Other suitable examples of antioxidants include, but are not limited to, alkylated monophenols, including but not limited to, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1-yl)phenol and mixtures thereof, alkylthiomethylphenols, including but not limited to, 2,4-dioctylthiorethyl-6-tert-hutylphenol, 2,4-dioctylthiomethyl-6-methylphenol, 2,4-dioetylthiomethyl-6-ethylphenol, 2,6-di-dodecylthiornethyl-4-nonylphenol, hydroquinones and alkylated hydroquinones, including but not limited to, 2,6-di-tert-hutyl-4-methoxyphenol, 2,5-di-tert-butylhydroquinone, 2,5-di-tort-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, tocopherols, including but not limited to, α-tocopherol, β-tocopherol, γ-tocopherol, δ-tocopherol and mixtures thereof (vitamin E), hydroxylated thiodiphenyl ethers, including but not limited to, 2,2′-thiobis(6-tort-butyl-4-methylphenol), 2,2′-thiobis(4-oetylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)-disulfide, alkylidenebisphenols, including but not limited to, 2, 2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-test-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecylmercaptobutane, 1,5,5-tetra-(5-tert-butyl-4-hydroxy-2-methylphenyl)pentane, O-, N- and S-benzyl compounds, including but not limited to, 3,5,3′,5′-tetra-tert-butyl.-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate, hydroxybenzylated malonates, including but not limited to, dioctadecyl-2,2-bis(3,5-di-tert-butyl-2-hydroxybenzyl)malonate, di-octadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate, aromatic hydroxybenzyl compounds, including but not limited to, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, triazine compounds, including but not limited to, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)iso-cyanurate, benzylphosphonates, including but not limited to, dimethyl-2,5-di-tert-butyl-4-hydroxybenzylphosphonate, diethyl-3,5-di-tert-butyl-4-hydroxybenzylphosphonate, dioctadecyl3,5-di-tent-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid, acylaminophenols, including but not limited to, 4-hydroxylauranilide, 4-hydroxystearanilide, octyl N-(3,5-di-tert-butyl-4-hydroxyphenyl)carbamate, esters of β-(3,5-di-tert-butyl-4-hydroxyphenyl)propionic acid with mono- or polyhydric alcohols, e.g. with methanol, ethanol n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, esters of β-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2] octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane, esters of 6-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, —N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2,2]octane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycal, thiodiethyl.ene glycol, diethylene glycol, triethylene glycol, pentaerythritol, tris(hydroxyethyl)isocyanurate, —N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, amides of 6-(3,5-di-tert-butyl-4-hydroxyphenyl)propionic acid e.g. N,N′-bis(3,5-di-tert-butylA-hydroxyphenylpropionyl)hexamethylenediamide, N,N′-bis(3,5-di-tert-butyl-4-hydroxyphenylpropionyl)trimethylenediamide, —N,N′-bis(3,5-di-tert-butyl-4-hydroxyphenylpropionyl)hydrazide, —N,N′-bis[2-(3-[3,5-di-tert-butyl-4-hydroxyphenyl]propionyloxy)ethyl]oxamide (Naugard®XL-1, supplied by Uniroyal), ascorbic acid (vitamin C), aminic antioxidants, including but not limited to, N,N′-di-isopropyl-p-phenylenediamine, —N,N′-di-sec-butyl-p-phenylenediamine, —N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenyienediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, including but not limited to, p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated teak-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene, and combinations of the foregoing.

In certain embodiments, suitable pharmaceutically acceptable excipients may include acrylics, cellulose derivatives, polysaccharides, monosaccharides, gums, natural or synthetic polymers (e.g., polyalkylene oxides (e.g., polymethylene oxides, polyethylene oxides, polypropylene oxides) polyethylenes, polypropylenes, polyvinyl chlorides, polycarbonates, polystyrenes, polyacrylates, polycaprolactone, polymethacrylates copolymers thereof, and mixtures thereof), liposomes, disintegrants (e.g., polyvinylpyrrolidone, sodium starch glycolate, crosscarmellose sodium, or a mixture thereof), glidants, lubricants, absorption enhancers, surfactants, binders, softeners, plasticizers (e.g., lecithin, hydrogenated vegetable oils, glycerol ester, lanolin, methyl ester, pentaerythritol ester, rice bran wax, stearic acid, sodium potassium stearates, and the like), waxes, fats, emulsifiers, fillers, antioxidants, colorants, diluents, processing aids (e.g., granulating aids), fixing agents (e.g., polyols such as, without limitations, sorbitol, maltitol/isomalt, mannitol, starch, and the like), pH-adjusting agents, viscosity adjusting agents, solubility increasing or descreasing agents, osmotic agents, solvents, or a combination thereof.

In certain embodiments, suitable pharmaceutically acceptable excipients may include polyvinylpyrrolidone, natural and synthetic gums, polyvinyl alcohol, corn starch, hydrophilic and hydrophobic materials such as sustained release polymers, acrylic resins, protein-derived materials, waxes, shellacs, and solid or semi-solid oils such as hydrogenated castor oil and hydrogenated vegetable oil. More specifically, the controlled release materials can be, e.g., alkylcelluloses such as ethylcellulose, acrylic and methacrylic acid polymers and copolymers (e.g., acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), glycidyl methacrylate copolymers, and mixtures of any of the foregoing), and cellulose ethers, such as hydroxyalkylcelluloses (e.g., hydroxypropylmethylcellulose) and carboxyalkylcelluloses. Waxes include, e.g., natural and synthetic waxes, fatty acids, fatty alcohols, and mixtures of the same (e.g., beeswax, carnauba wax, stearic acid and stearyl alcohol).

In certain embodiments, suitable pharmaceutically acceptable excipients may include gelling agents, such as and without limitation, sugars or sugar derived alcohols, such as mannitol, sorbitol, and the like, starch and starch derivatives, cellulose derivatives (such as microcrystalline cellulose, sodium caboxymethyl cellulose, methylcellulose, ethyl cellulose, hydroxyethyl cellulose, hydroxypropyl cellulose, hydroxypropyl methylcellulose, cellulose esters, cellulose diesters, cellulose triesters, cellulose ethers, cellulose ester-ethers, cellulose acylates, cellulose diacylates, cellulose triacylates, cellulose acetates, cellulose diacetates, cellulose triacetates, cellulose acetate propionates, cellulose acetate butyrates, cellulose acetate succinate, cellulose acetate phthalate, hydroxypropyl methyl cellulose phthalate, hydroxy propyl methyl cellulose acetate succinate (hypermellose acetate succinate), and mixtures thereof), attapulgites, bentonites, dextrins, alginates, algenic acid salts such as sodium alginate and potassium alginate, casein, stearic acid, shellac, carrageenan, gum tragacanth, gum acacia, gum arabic, pullulan gum, dextrin, gellan gum, agar gum, tara gum, karaya, guar gum, welan gum, rhamsan gum, locust bean gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof.

In certain embodiments, suitable pharmaceutically acceptable excipients may include hydrophilic excipients, such as without limitations, water, low molecular weight polyols, such as, polyethylene glycol, polypropylene glycol, or a combination thereof. Examples of other suitable hydrophilic carriers include, without limitations, polyoxyethylene derivatives of a sorbitan ester, such as sorbitan monolaurate (Polysorbate 20), Polysorbate 80, Polysorbate 60, polyoxyethylene 20 sorbitan trioleate (Polysorbate 85), acetic acid, formic acid, other hydrophilic surfactants and mixtures thereof. Exemplary low molecular weight polyols include, without limitations, those having a number average molecular weight of from any of about 200 Dalton, about 400 Dalton, about 600 Dalton, about 800 Dalton, or about 1000 Dalton to any of about 2000 Dalton, about 3000 Dalton, about 4000 Dalton, about 5000 Dalton, about 6000 Da, or about 7000 Da, or any sub-range or single value therein (for instance, polyethylene glycol 400, polyethylene glycol 600, or the like).

In certain embodiments, suitable pharmaceutically acceptable excipients may include plasticizers, such as, but not be limited to, sugar alcohol plasticizer such as triacetin, isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In certain embodiments, suitable pharmaceutically acceptable excipients may include plasticizer such as, without limitations, phosphate esters; phthalate esters; amides; mineral oils; fatty acids and esters; fatty alcohols, vegetable oils and hydrogenated vegetable oils including acetylated hydrogenated cottonseed glyceride and acetylated hydrogenated soybean oil glycerides; acetyl tributyl citrate, acetyl triethyl citrate, Castor oil, diacetylated monoglycerides, dipropylene glycol salicylate glycerin, glyceryl cocoate, mono- and di-acetylated monoglycerides, nitrobenzene, carbon disulfide, fl-naphtyl salicylate, phthalyl glycolate, diocyl phthalate; sorbitol, sorbitol glyceryl tricitrate; sucrose octaacetate; a-tocopheryl polyethylene glycol succinate, phosphate esters; phthalate esters; amides; mineral oils; fatty acids and esters; fatty alcohols; and vegetable oils, fatty alcohols including cetostearyl alcohol, cetyl alcohol, stearyl alcohol, oleyl alcohol and myristyl alcohol; methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate, butyl oleate, butyl stearate, di(beta-methoxyethyl) adipate, dibutyl sebacate, dibutyl tartrate, diisobutyl adipate, dihexyl adipate, triethylene glycol di(beta-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, triethylene glycol dipelargonate, beta-(p-tert-amyl phenoxy)ethanol, beta(p-tert-butytphenoxy)ethanol, beta-(p-tert-butytphenoxyethyl)acetate, bis(beta-p-tert-buthylphenoxydiethyl)ether, camphor, Cumar W-1, Cumar MH-1, Cumar V-1, diamyl phthalate, (diamylphenoxy) ethanol, diphenyl oxide, technical hydroabietyl alcohol, beckolin, benzene hexahydrochlonde, Clorafin 40, Piccolastic A-5, Piccalastic A-25, Flexol B-400, Glycerol alfa-methyl alfa-phenyl ether, chlorinated naphthalene, HB-40, monoamylphthalate. Nevillac 10 o-nitrodiphenyl and Paracril 26.

In certain embodiments, suitable pharmaceutically acceptable excipients may include plasticizer such as, without limitations, sugar alcohol plasticizer such as isomalt, maltitol, sorbitol, xylitol, erythritol, adonitol, dulcitol, pentaerythritol, or mannitol; or polyol plasticizer such as glycerin, diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In certain embodiments, suitable pharmaceutically acceptable excipients may include fragrances such as, without limitations, natural and/or synthetic fragrance raw materials. For instance, oil soluble perfume oils, which may or may not be in mixture with water soluble perfume oils. Oil soluble perfume materials are natural, or natural-identical essential oils such as orange oil, lavender oil, pine oil, eucalyptus oil, lemon oil, clove leaf, peppermint oil, cedarwood oil, rosemary oil, bergamot oil, lavandin oil, patchouli oil, chamomile oil, jasmine oil, spike oil, rose oil, Vetiver oil, fennel oil, anise oil, thyme oil, germanium oil, menthol, and marjoram oil. An animal fragrance is for example musk, castoreum, aber or zibet. Spagyric essences are also known in the art. They are made by fermenting certain herbs that are then processed to the final product. Synthetic fragrance ingredients are for example synthetic essential oils such as composed of single compounds such as linalol, terpineol, nerol, citronellal, benzaldehyde, cinnamon aldehyde, vanillin, ethylvanillin, or methylacetophenone. The fragrance materials may also be synthetic oil soluble perfume oils selected from the usual group consisting of fragrant hydrocarbons, alcohols, ketones, aldehydes, ethers, esters, polyene derivatives. Other fragrances that may be used are catalogued and described in references and databases such as S. Arctander, Perfume and Flavor Chemicals, Volumes I and II (1960, 1969; reprint 2000); Allured's Flavor and Fragrance Materials (2005); and database maintained by the Research Institute for Fragrance Materials at www.rifm.org.

In certain embodiments, suitable pharmaceutically acceptable excipients may include a perfume oil. Suitable perfume oils include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include mainly the terpenes and balsams.

In certain embodiments, suitable pharmaceutically acceptable excipients may include essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, suitable pharmaceutically acceptable excipients may include preservatives. The term “preservative”, as used herein, refers to an agent that extends the storage life of the dosage form by retarding or preventing deterioration of flavor, odor, color, texture, appearance, therapeutic value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involve mode of action (a preservative prevents growth rather than killing microorganisms) and exposure time (a preservative has days to months to act whereas a sanitizer has at most a few minutes to act). Suitable preservatives include, without limitations, phenoxyethanol, a solution of paraben, pentanediol and sorbic acid, as well as silver complexes.

In certain embodiments, suitable pharmaceutically acceptable excipients may include coloring agents, such as, without limitations, colors such as e.g., white, black, yellow, blue, green, pink, red, orange, violet, indigo, and brown.

In certain embodiments, suitable pharmaceutically acceptable excipients may include alkalizing agent(s), such as, without limitations, magnesium oxide, ammonium hydroxide, sodium hydroxide, sodium carbonate, sodium citrate, trisodium phosphate and/or disodium phosphate.

In certain embodiments, suitable pharmaceutically acceptable excipients may include lubricant(s)/release agent(s) such as, but not limited to, fatty acids and their salts, fatty alcohols, fatty esters, fatty amines, fatty amine acetates and fatty amides. Other suitable lubricants may include, but not be limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

In certain embodiments, suitable pharmaceutically acceptable excipients may include diluents such as, but not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

In certain embodiments, suitable pharmaceutically acceptable excipients may include oils and fats such as, but not be limited to, almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil. Other oil and fats that may be in the fill of the PVA shell may include, but not be limited to, fish oil (omega-3), crill oil, animal or vegetable fats, e.g., in their hydrogenated form, mono-, di-, and tri-glycerides with C12-, C14-, C16-, C18-, C20- and C22-fatty acids.

In certain embodiments, suitable pharmaceutically acceptable excipients may include vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylamides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers; inorganic fillers, such as the oxides of magnesium aluminum, silicon, titanium, etc.

In certain embodiments, suitable pharmaceutically acceptable excipients may include a hydrophobic material, including, but not limited to, digestible, long chain (C₈-C₅₀, especially C₁₂-C₄₀), substituted or unsubstituted hydrocarbons, such as natural or synthetic waxes (such as beeswax, glycowax, castor wax and carnauba wax), fatty alcohols (such as lauryl, myristyl, stearyl, cetyl or preferably cetostearyl alcohol), fatty acids, including, but not limited to, mono-diglyceride of medium chain fatty acids (such as caprylic, capric, caproic, lauric, oleic, linoleic), medium chain triglycerides, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

In certain embodiments, suitable pharmaceutically acceptable excipients may include polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, acetic acid, caprylic acid, oleic acid, polyaminoacids or peptides, polyamides, polyacrylamide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, PEG-35 castor oil, caprylocaproyl polyoxyl-8 glycerides, glyceryl distearate, and combinations thereof.

In certain embodiments, suitable pharmaceutically acceptable excipients may include surfactants such as, without limitations, polysorbate 80-polyoxyethylene (20) sorbitan monooleate, polyoxyl 40 hydrogenated castor oil, polyoxyl 35 castor oil, caprylocaproyl macrogol glycerides, and combinations thereof.

In certain embodiments, suitable pharmaceutically acceptable excipients may include fillers such as, without limitations, lactose, microcrystalline cellulose, and combinations thereof.

In certain embodiments, suitable pharmaceutically acceptable excipients may include natural gums (e.g., a natural plant gum). Suitable natural gums include, without limitations, guar gum, carob gum, konjac gum, xanthan gum, sclerotium gum, acacia gum, cellulose gum (modified or not), or a combination thereof.

In certain embodiments, suitable pharmaceutically acceptable excipients may include emulsifiers such as, without limitations, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate, or a combination thereof.

Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodium cetearyl sulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobutene. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

In certain embodiments, suitable pharmaceutically acceptable excipients may include chelating agents such as, without limitations, disodium ethylenediaminetetraacetic acid (EDTA), diethylenetriaminepentaacetic acid (DTPA), N-(hydroxyethyl)-ethylenediaminetriacetic acid (HEDTA), and nitrilotriacetic acid (NTA).

In certain embodiments, suitable pharmaceutically acceptable excipients may include fatty alcohols, such as, without limitations guerbet alcohols based on fatty alcohols having from 6 to 18, preferably from 8 to 10 carbon atoms including cetyl alcohol, stearyl alcohol, cetearyl alcohol, oleyl alcohol, octyldodecanol, benzoate of C₁₂-C₁₅ alcohols, acetylated lanolin alcohol, etc.

In certain embodiments, suitable pharmaceutically acceptable excipients may include esters of fatty acids, such as, without limitations esters of linear C₆-C₂₄ fatty acids with linear C₃-C₂₄ alcohols, esters of branched C₆-C₁₃carboxyl acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and technical-grade mixtures thereof (obtained, for example, in the high-pressure hydrogenation of technical-grade methyl esters based on fats and oils or aldehydes from Roelen's oxosynthesis and as monomer fractions in the dimerization of unsaturated fatty alcohols). Additional suitable examples of ester oils are isopropyl myristate, isopropyl palmitate, isopropyl stearate, isopropyl isostearate, isopropyl oleate, n-butyl stearate, n-hexyl laurate, n-decyl oleate, isooctyl stearate, iso-nonylstearate, isononyl isononanoate, 2-ethylhexylpalmitate, 2-hexyllaurate, 2-hexyldecylstearate, 2-octyldodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, etc.

In certain embodiments, suitable pharmaceutically acceptable excipients may include other adjuvants, such as, without limitations, diethylhexyl 2,6-naphthalate, di-n-butyl adipate, di(2-ethylhexyl)-adipate, di(2-ethyl hexyl)-succinate and diisotridecyl acelaat, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. Esters of C₆-C₂₄ fatty alcohols and/or Guerbet alcohols with aromatic carboxylic acids, saturated and/or unsaturated, especially benzoic acid, esters of C₂-C₁₂ dicarboxylic acids with linear or branched alcohols having from 1 to 22 carbon atoms or polyols having from 2 to 10 carbon atoms and from 2 to 6 hydroxy groups.

In certain embodiments, suitable pharmaceutically acceptable excipients may include natural or synthetic triglycerides (including glyceryl esters and derivatives), such as, without limitations, di- or triglycerides, based on C6-Cis fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc. Additional suitable excipients include waxes including esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides.

In certain embodiments, suitable pharmaceutically acceptable excipients may include pearlescent waxes, such as, without limitations, alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially lauryl and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

In certain embodiments, suitable pharmaceutically acceptable excipients may include hydrocarbon oils, such as, without limitations, mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant and animal kingdom.

In certain embodiments, suitable pharmaceutically acceptable excipients may include silicones or siloxanes (organosubstituted polysiloxane), such as, without limitations, dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form. Linear polysiloxanes, dimethicone (Dow Corning 200 fluid, Rhodia Mirasil DM), dimethiconol, cyclic silicone fluids, cyclopentasiloxanes volatiles (Dow Corning 345 fluid), phenyltrimethicone (Dow Corning 556 fluid). Also suitable are simethicones, which are mixtures of dimethicones having an average chain length of from 200 to 300 dimethylsiloxane units with hydrogenated silicates. A detailed survey by Todd et al. of suitable volatile silicones may in addition be found in Cosm. Toil. 91, 27 (1976).

In certain embodiments, suitable pharmaceutically acceptable excipients may include emulsifiers, such as, without limitations, carboxylic acids and their salts: alkaline soap of sodium, potassium and ammonium, metallic soap of calcium or magnesium, organic basis soap such as Lauric, palmitic, stearic and oleic acid etc. Alkyl phosphates or phosphoric acid esters, acid phosphate, diethanolamine phosphate, potassium cetyl phosphate. Ethoxylated carboxylic acids or polyethylene glycol esters, PEG-n acylates. Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol polyglycol ether such as laureth-n, ceteareth-n, steareth-n, oleth-n. Fatty acid polyglycolether such as PEG-n stearate, PEG-n oleate, PEG-n cocoate. Monoglycerides and polyol esters. C₁₂-C₂₂ fatty acid mono- and di-esters of addition products of from 1 to 30 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono- and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C8-C22 alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated paraffins, sulfonated tetrapropyene sulfonate, sodium lauryl sulfates, ammonium and ethanolamine lauryl sulfates, lauryl ether sulfates, sodium laureth sulfates, sulfosuccinates, acetyl isothionates, alkanolamide sulfates, taurines, methyl taurines, imidazole sulfates. Polysiloxane/polyalkyl/polyether copolymers and derivatives, dimethicone, copolyols, silicone polyethylene oxide copolymer, silicone glycol copolymer. Propoxylated or POE-n ethers (Meroxapols), Polaxamers or poly(oxyethylene)m-block-poly(oxypropylene)n-block(oxyethylene). Zwitterionic surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkyl betaine, N-alkylaminobetaines. Alkylimidazolines, alkylopeptides, lipoaminoacides, self-emulsifying bases and the compounds as described in K. F. DePolo, A short textbook of cosmetology, Chapter 8, Table 8-7, p 250-251.

Suitable nonionic bases include, without limitations, PEG-6 beeswax (and) PEG-6 stearate (and) polyglyceryl-2-isostearate, glyceryl stearate (and) PEG-100 stearate, PEG-5 glyceryl stearate, sorbitan oleate (and) polyglyceryl-3 ricinoleate, sorbitan stearate and sucrose cocoate, glyceryl stearate and laureth-23, cetearyl alcohol and ceteth-20, cetearyl alcohol and polysorbate 60 and PEG-150 and stearate-20, cetearyl alcohol and cetearyl polyglucoside, cetearyl alcohol and ceteareth-20, cetearyl alcohol and PEG-40 castor oil, cetearyl alcohol and PEG-40 castor oil and sodium cetearyl sulfate, stearyl alcohol and steareth-7 and steareth-10, cetearyl alcohol and szeareth-7 and steareth-10, glyceryl stearate and PEG-75 stearate, propylene glycol ceteth-3 acetate, propylene glycol isoceth-3 acetate, cetearyl alcohol and ceteth-12 and oleth-12, PEG-6 stearate and PEG-32 stearate, PEG-6 stearate and ceteth-20 and steareth-20, PEG-6 stearate and ceteth-20 and glyceryl stearate and steareth-20, glyceryl stearate and ceteareth-20.

Suitable anionic alkaline bases includes, without limitations, PEG-2 stearate SE, glyceryl stearate SE, propylene glycol stearate. Anionic acid bases such as cetearyl Alcohol and Sodium cetearyl sulfate, cetearyl alcohol and sodium lauryl sulfate, trilaneth-4 phosphate and glycol stearate and PEG-2 stearate, glyceryl stearate and sodium lauryl Sulfate. Cationic acid bases such as cetearyl alcohol and cetrimonium bromide.

In certain embodiments, suitable pharmaceutically acceptable excipients may include adjuvants and additives, such as, without limitations, surfactants, super-fatting agents, consistency regulators, thickeners, polymers, stabilizers, biogenic active ingredients, swelling agents, further UV light-protective factors, antioxidants, hydrotropic agents, preservatives, self-tanning agents, solubilizers, perfume oils, colorants, bacteria-inhibiting agents and the like.

In certain embodiments, suitable pharmaceutically acceptable excipients may include super-fatting agents, such as, without limitations, lanolin and lecithin and also polyethoxylated or acetylated lanolin and lecithin derivatives, polyol fatty acid esters, monoglycerides and fatty acid alkanolamides, the latter simultaneously acting as foam stabilizers.

In certain embodiments, suitable pharmaceutically acceptable excipients may include surfactants, such as, without limitations, fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, alpha.-olefin sulfonates, ethercarboxylic acids, alkyl oligoglucosides, fatty acid glucamides, alkylamidobetaines and/or protein fatty acid condensation products, the latter preferably being based on wheat proteins.

In certain embodiments, suitable pharmaceutically acceptable excipients may include consistency regulators/thickeners and rheology modifiers, such as, without limitations, silicium dioxide, magnesium silicates, aluminium silicates, polysaccharides or derivatives thereof for example hyaluronic acid, xanthan gum, guar-guar, agar-agar, alginates, carrageenan, gellan, pectines, or modified cellulose such as hydroxycellulose, hydroxypropylmethylcellulose. In addition polyacrylates or homopolymer of reticulated acrylic acids and polyacrylamides, carbomer (CARBOPOL types 980, 981, 1382, ETD 2001, ETD2020, ULTREZ 10) or SALCARE range such as SALCARE SC80 (steareth-10 allyl ether/acrylates copolymer), Salcare SC81 (acrylates copolymer), Salcare SC91 and Salcare AST (sodium acrylates copolymer/PPG-1 trideceth-6), SEPIGEL 305 (polyacrylamide/laureth-7), SIMULGEL NS and SIMULGEL EG (hydroxyethyl acrylate/sodium acryloyldimethyl taurate copolymer), STABILEN 30 (acrylates/vinyl isodecanoate crosspolymer), PEMULEN TR-1 (acrylates/C10-30 alkyl acrylate crosspolymer), LUVIGEL EM (sodium acrylates copolymer), ACULYN 28 (acrylates/beheneth-25 methacrylate copolymer), etc.

In certain embodiments, suitable pharmaceutically acceptable excipients may include polymers, such as, without limitations, an anionic, zwitterionic, amphoteric and non-ionic polymers there come into consideration, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyl-trimethylammonium chloride/acrylate copolymers, octyl acrylamide/methyl methacrylate-tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatized cellulose ethers and silicones. Furthermore, the polymers as described in EP 1093796 (pages 3-8, paragraphs 17-68) may be used.

In certain embodiments, suitable pharmaceutically acceptable excipients may include antioxidants, such as, without limitations amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl)propionyl]sulfanilic acid (and salts thereof, for example the disodium salts), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS(=“Hindered Amine Light Stabilizers”) compounds may also be mentioned.

In certain embodiments, suitable pharmaceutically acceptable excipients may include hydrotropic agents, such as, without limitations, ethoxylated or non-ethoxylated mono-alcohols, diols or polyols with a low number of carbon atoms or their ethers (e.g. ethanol, isopropanol, 1,2-dipropanediol, propylene glycol, glycerin, ethylene glycol, ethylene glycol monoethylether, ethylene glycol monobutylether, propylene glycol monomethylether, propylene glycol monoethylether, propylene glycol monobutylether, diethylene glycol monomethylether; diethylene glycol monoethylether, diethylene glycol monobutylether and similar products). The polyols that come into consideration for that purpose have preferably from 2 to 15 carbon atoms and at least two hydroxy groups. The polyols may also contain further functional groups, especially amino groups, and/or may be modified with nitrogen. Typical examples are as follows: glycerol, alkylene glycols, for example ethylene glycol, diethylene glycol, propylene glycol, butylene glycol, hexylene glycol and also polyethylene glycols having an average molecular weight of from 100 to 1000 Dalton; technical oligoglycerol mixtures having an intrinsic degree of condensation of from 1.5 to 10, for example technical diglycerol mixtures having a diglycerol content of from 40 to 50% by weight; methylol compounds, such as, especially, trimethylolethane, trimethylolpropane, trimethylolbutane, pentaerythritol and dipentaerythritol; lower alkyl-glucosides, especially those having from 1 to 8 carbon atoms in the alkyl radical, for example methyl and butyl glucoside; sugar alcohols having from 5 to 12 carbon atoms, for example sorbitol or mannitol; sugars having from 5 to 12 carbon atoms, for example glucose or saccharose; amino sugars, for example glucamine; dialcohol amines, such as diethanolamine or 2-amino-1,3-propanediol.

In certain embodiments, suitable pharmaceutically acceptable excipients may include preservatives, such as, without limitations, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan and further substance classes listed in the following reference: K. F. DePolo-A short textbook of cosmetology, Chapter 7, Table 7-2, 7-3, 7-4 and 7-5, p 210-219.

In certain embodiments, suitable pharmaceutically acceptable excipients may include bacteria-inhibiting agents, such as, without limitations, 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-trimethyl-2,6,10-dodecatrien-1-ol), which is present in lime blossom oil. Glycerol monolaurate has also proved to be a bacteriostatic agent.

In certain embodiments, pharmaceutically acceptable excipients may be included (individually or cumulatively) in the pharmaceutical compositions described herein in a concentration ranging from any of about 5 wt %, about 10 wt %, about 15 wt %, about 20 wt %, about 25 wt %, about 30 wt %, about 35 wt %, about 40 wt %, about 45 wt %, or about 50 wt % to any of about 55 wt %, about 60 wt %, about 65 wt %, about 70 wt %, about 75 wt %, about 80 wt %, about 85 wt %, about 90 wt %, about 95 wt %, or about 99 wt %, or any sub-range or single value therein based on the total weight of the composition.

### Method of Preparation

In certain embodiments, the instant disclosure is directed to a method of preparing any of the nasal compositions described herein. In certain embodiments, the method includes combining a therapeutically effective amount of the compounds disclosed herein with one or more nasal pharmaceutically acceptable excipients and containing the formulation in a container or device suitable for nasal delivery.

### Additional Active Agents and Embodiments

In certain embodiments, the present invention is directed to a method of treating opioid overdose or prophylactically treating opioid overdose comprising administering an opioid receptor antagonist and a respiratory stimulant (e.g., of formula I) to a patient in need thereof.

In another aspect, the present invention is directed to a nasal pharmaceutical composition or system (formulation in a device) comprising a combination of an opioid receptor antagonist and a respiratory stimulant (e.g., of formula I), wherein the combination is in an effective amount to treat or prophylactically treat opioid overdose in a patient in need thereof.

In certain embodiments, the present invention is directed to a method of treating opioid withdrawal symptoms or prophylactically treating opioid withdrawal symptoms comprising administering an opioid receptor antagonist and a respiratory stimulant (e.g., of formula I) to a patient in need thereof.

In another aspect, the present invention is directed to a nasal pharmaceutical composition or system (formulation in a device) comprising a combination of an opioid receptor antagonist and a respiratory stimulant (e.g., of formula I), wherein the combination is in an effective amount to treat or prophylactically treat opioid withdrawal symptoms in a patient in need thereof.

In a particular aspect, the opioid antagonist is selected from the group consisting of naltrexone, naloxone, nalmefene, and a pharmaceutically-acceptable salt thereof.

In one aspect, the weight ratio of the antagonist to the respiratory stimulant can be from about 1:9 to about 9:1, from about 1:7 to about 7:1, from about 1:5 to about 5:1, or from about 1:3 to about 3:1.

In some aspects of a nasal formulation, the pharmaceutical composition of the respiratory stimulant and the optional antagonist includes one or more excipients selected from water, NaCl, benzalkonium chloride, sodium edetate, disodium edetate, and hydrochloric acid.

In some aspects of a nasal formulation, the pharmaceutical composition further comprises water, NaCl, benzalkonium chloride, disodium edetate, and hydrochloric acid.

In some aspects of a nasal formulation, the pharmaceutical composition further comprises: an isotonicity agent; a preservative; a stabilizing agent; an amount of an acid sufficient to achieve a pH or 3.5-5.5; and an amount of water.

In some aspects of a nasal formulation, the pharmaceutical composition comprises: between about 0.2 mg and about 1.2 mg of an isotonicity agent; between about 0.005 mg and about 0.015 mg of a preservative; between about 0.1 mg and about 0.5 mg of a stabilizing agent; an amount of an acid sufficient to achieve a pH or 3.5-5.5; and an amount of water.

In some aspects of a nasal formulation, the isotonicity agent is NaCl; the preservative is benzalkonium chloride; the stabilizing agent is disodium edetate; and the acid is hydrochloric acid.

In some aspects of a nasal formulation, said pharmaceutical composition comprises: about 0.74 mg NaCl; about 0.01 mg benzalkonium chloride; about 0.2 mg disodium edetate; an amount of hydrochloric acid sufficient to achieve a pH or 3.5-5.5; and an amount of water.

In one aspect of a nasal formulation, the antagonist is naloxone in an amount equivalent to about 4 mg to about 10 mg of naloxone hydrochloride. In some aspects, said therapeutically effective amount is equivalent to an amount chosen from about 2 mg naloxone hydrochloride, about 4 mg of naloxone hydrochloride, and about 8 mg naloxone hydrochloride.

In some aspects of a nasal formulation, the naloxone amount is equivalent to about 2 mg of naloxone hydrochloride. In some aspects of a nasal formulation, the naloxone amount is equivalent to about 4 mg of naloxone hydrochloride. In some aspects of a nasal formulation, the naloxone amount is equivalent to about 8 mg of naloxone hydrochloride. In some aspects of a nasal formulation, the naloxone amount is equivalent to about 3.4 mg of naloxone hydrochloride.

In some aspects of a nasal formulation, the naloxone amount is about 2.2 mg to about 13.2 mg of naloxone hydrochloride dihydrate. In some aspects of a nasal formulation, the naloxone amount is about 4.4 mg to about 11 mg of naloxone hydrochloride dihydrate. In some aspects, the naloxone amount is an amount chosen from about 2.2 mg naloxone hydrochloride dihydrate, about 4.4 mg of naloxone hydrochloride dihydrate, and about 8.8 mg naloxone hydrochloride dihydrate. In some aspects of a nasal formulation, the naloxone amount is about 2.2 mg of naloxone hydrochloride dihydrate. In some aspects of a nasal formulation, the naloxone amount is about 4.4 mg of naloxone hydrochloride dihydrate. In some aspects of a nasal formulation, the naloxone amount is about 8.8 mg of naloxone hydrochloride dihydrate.

In some aspects of a nasal formulation, the pharmaceutical composition comprises: between about 0.2 mg and about 1.2 mg of an isotonicity agent; between about 0.005 mg and about 0.015 mg of a preservative; between about 0.1 mg and about 0.5 mg of a stabilizing agent; an amount of an acid sufficient to achieve a pH or 3.5-5.5; and an amount of water.

In some aspects of a nasal formulation, the isotonicity agent is NaCl; the preservative is benzalkonium chloride; the stabilizing agent is disodium edetate; and the acid is hydrochloric acid.

In some aspects of a nasal formulation, said pharmaceutical composition comprises: about 0.74 mg NaCl; about 0.01 mg benzalkonium chloride; about 0.2 mg disodium edetate; an amount of hydrochloric acid sufficient to achieve a pH or 3.5-5.5; and an amount of water.

The drug of abuse that is the subject of the treated overdose can be an opioid, for example, morphine; codeine; thebaine; oripavine; diacetylmorphine; 2,4-dinitrophenylmorphine; methylenedioxydimethylamphetamine; chlomaltrexamine; dihydromorphine; hydromorphinol; nicomorphine; dipropanoylmorphine; desomorphine; acetylproprionylmorphine; methyldesorphine; N-phenethylnormorphine; 14-hydroxydihydrocodeine (RAM-318); 7,8-dihydro-14-hydroxy-N-phenethylnormorphine (RAM-378); dibenzoylmorphine; diacetyldihydromorphine; dibenzoylmorphine; 6-monoacetylcodeine (6-MAC); acetyldihydrocodeine; dihydrocodeine; nalbuphine; nicocodeine; nicodicodeine; oxymorphazone; 1-iodomorphine; morphine-6-glycuronide (M6G); 6-monoacetylmorphine (6-MAM); norcodeine; normorphine; genomorphine; dextrallorphan (DXA); cyclorphan; dihydroheterocodeine; pholcodine; myrophine; 14-cinnamoyloxycodeinone; 14-ethoxymetopon; 14-methoxymetopon; 14-phenylpropoxymetopon (PPOM); 7-spiroindanyloxymorphone; acetylmorphone; codeinone; conorphone; codoxime; thebacon; metopon; N-phenethyl-14-ethoxymetopon; morphinone; benzylmorphine; codeine methylbromide; ethylmorphine; heterocodeine; hydromorphone; hydrocodone; oxycodone; oxymorphone; pentamorphone; semorphone; chloromorphide; ethylmorphine; buprenorphine; fentanyl; alphamethylfentanyl; alfentanil; sufentanil; remifentanil; carfentanyl; ohmefentanyl; pethidine; ketobemidone; desmethylprodine (MPPP); allylprodine; prodine; 1-methyl-4-phenyl-4-propionoxypiperidine (PEPAP); propoxyphene; dextropropoxyphene; dextromoramide; bezitramide; piritramide; levorphanol; methadone; dipipanone; levomethadyl acetate (LAAM); difenoxin; diphenoxylate; loperamide; dezocine; pentazocine; phenazocine; dihydroetorphine; etorphine; butorphanol; nalbuphine; levomethorphan; levophenacylmorphan; norlevorphanol; oxilorphan; phenomorphan; furethylnorlevorphanol; xorphanol; butorphanol; cyprodime; drotebanol; 7-PET; acetorphine; BU-48; cyprenorphine; norbuprenorphine; lefetamine; meptazinol; mitragynine; tilidine; tramadol; tapentadol; dextropropoxyphene; endorphins; enkephalins; dynorphins; and endomorphins. In some embodiments, the opioid is oxymorphone. In further embodiments, the opioid is oxycodone. The above list of opioids also includes pharmaceutically acceptable salts thereof.

In a particular aspect, the respiratory stimulant can be selected from the group consisting of doxapram, almitrine, a compound of formula I and a pharmaceutically-acceptable salt thereof.

### Devices

In certain embodiments are provided nasal drug delivery devices comprising a pharmaceutical composition described herein. The benefits of nasal delivery includes a needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

The liquid nasal formulations may be aqueous solutions, suspensions or emulsions. In certain spray pump systems, antimicrobial preservatives may be included to maintain microbiological stability.

In certain embodiments, the nasal formulations disclosed herein can be a metered spray pump.

The pumps can deliver, e.g., 0.05 ml to about 0.5 mL or about 0.1 mL. In certain embodiments, the formulations include a preservative and in other embodiments the formulation does not include a preservative. In certain embodiments, a spray pump of the present invention replaces the emitted liquid with air, and preservatives are therefore included to prevent contamination. In other embodiments, systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a headdown application is recommended. Another method is that the air that replaces the emitted liquid is filtered through an aseptic air filter. In addition, some systems have a ball valve at the tip to prevent contamination of the liquid inside the applicator tip.

In certain embodiments, metered-dose spray pumps require priming and some degree of overfill to maintain dose conformity for the labeled number of doses. In other embodiments, single-dose or bi-dose spray devices are preferred.

In certain embodiments, the systems are terminally sterilized. Products may be filled and sealed in a controlled environment to minimize the microbial and particulate content of the in-process product and to help ensure that the subsequent sterilization process is successful. In certain embodiments, the product in its final container is then subjected to a sterilization process such as heat or irradiation. In an aseptic process, the drug product, container, and closure may be first subjected to sterilization methods separately, as appropriate, and then brought together.

### Pharmacodynamics

In some embodiments, the patient is free from respiratory depression for at least about half hour, at least about 1 hour, at least about 2 hours, at least about 3 hours, at least about 4 hours, at least about 6 hours or at least about 8 hours following treatment comprising delivery of the therapeutically effective amount of the respiratory stimulant and the optional antagonist.

In some embodiments, said device is a bi-dose device, wherein a first volume of the pharmaceutical composition is present in a first reservoir and a second volume of the pharmaceutical composition is present in a second reservoir, and wherein the therapeutically effective amount is delivered essentially by a first actuation of the device into a first nostril of the patient and a second actuation of the device into the same or a second nostril of the patient.

In certain embodiments with two agents, each formulation in the bi-dose device can be in both reservoirs in combination and other embodiments each active agent is separate in each reservoir.

In some embodiments, upon nasal delivery of the pharmaceutical composition to the patient, less than about 20%, less than about 10% or less than about 5% of the pharmaceutical composition leaves the nasal cavity via drainage into the nasopharynx or externally.

### EXAMPLES

Specific embodiments of the invention will now be demonstrated by reference to the following examples. It should be understood that these examples are disclosed solely by way of illustrating the invention and should not be taken in any way to limit the scope of the present invention.

### Stability Study

Formulations were prepared with formulations presented in Table 1. The formulations included the hydrogen sulfate salt of Compound A as an active agent (N-(4,6-Bis-n-propylamino-[1,3,5]triazin-2-yl)-O,N-dimethyl-hydroxylamine):

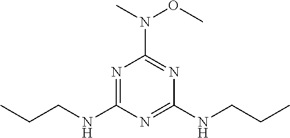 [SMILES: CCCNc1nc(NCCC)nc(N(C)OC)n1]

TABLE 1

| | | Concentration | |
|---|---|---|---|
| Sample | Formulations | mg/mL | pH |
| 1A | 10% v/v EtOH, 40% propylene | 20 | 4.5 |
| | glycol Citrate buffer | | |
| 1B | 10% v/v EtOH, 40% propylene | | |
| | glycol Acetate buffer | | |
| 2A | 10% v/v EtOH, 40% HP-β-CD | | |
| | Citrate buffer | | |
| 2B | 10% v/v EtOH, 40% HP-β-CD | | |
| | Acetate buffer | | |
| 3A | 10% v/v EtOH, 40% Kolliphor | | |
| | HS15 Citrate buffer | | |
| 3B | 10% v/v EtOH, 40% Kolliphor | | |
| | HS15 Acetate buffer | | |

The stability of the samples in Table 1 were tested after 4 weeks at 25° C. and 40° C. The results of which are presented in Table 2 below. It is noted that the concentration in Table 2 is that of the filtered concentration.

TABLE 2

| | Initial Time Point | | | 4 weeks at 25° C. | | | 4 weeks at 40° C. | | |
|---|---|---|---|---|---|---|---|---|---|
| | Conc | Purity | | Conc | Purity | | Conc | Purity | |
| Sample | mg/mL | area % | pH | mg/mL | area % | pH | mg/mL | area % | pH |
| 1A | 20.7 | 100.0 | 4.60 | 19.2 | 100.0 | 4.60 | 20.5 | 100.0 | 4.57 |
| 1B | 20.4 | 100.0 | 4.70 | 21.8 | 100.0 | 4.77 | 21.4 | 100.0 | 4.77 |
| 2A | 19.0 | 100.0 | 4.49 | 19.9 | 100.0 | 4.46 | 19.9 | 100.0 | 4.44 |
| 2B | 18.0 | 100.0 | 4.55 | 19.8 | 100.0 | 4.57 | 19.7 | 99.89 | 4.51 |
| | | | | | | | | RRT | |
| | | | | | | | | 1.35 = 0.11 | |
| 3A | 19.6 | 100.0 | 4.71 | 22.5 | 100.0 | 4.65 | 22.7 | 100.0 | 4.67 |
| 3B | 20.0 | 100.0 | 4.73 | 20.5 | 100.0 | 4.65 | 21.8 | 100.0 | 4.69 |

Another embodiment having a 15 mg/mL concentration was prepared and the stability was tested over the course of 2 weeks at 40° C. and 60° C. A 15 mg/mL concentration was prepared using 50% polyethylene glycol at a pH of 4.0 and 5.0. The results of the stability study is presented in Table 3-5.

TABLE 3
Results at Initial Time Point

| | Assay | | Impurities | Total |
|---|---|---|---|---|
| Sample | (mg/mL) | pH | present | Impurity |
| 50% PEG300 | 14.14 | 3.89 | RRT 0.70-0.71 = | 0.08 |
| pH 4.0 at 40° C. | | | 0.08 | |
| 50% PEG300 | 14.57 | 4.9 | RRT 0.70-0.71 = | <0.05 |
| pH 5.0 at 40° C. | | | <0.05 | |

TABLE 4
Results at 1 week

| | Assay | | Impurities | Total |
|---|---|---|---|---|
| Sample | (mg/mL) | pH | present | Impurity |
| 50% PEG300 | 14 | 3.87 | RRT 0.70-0.71 = 0.13 | 0.13 |
| pH 4.0 at 40° C. | | | | |
| 50% PEG300 | 13.8 | 3.74 | RRT 0.61-.062 = 0.43 | 1.43 |
| pH 4.0 at 60° C. | | | RRT 0.70-0.71 = 0.6 | |
| | | | RRT 0.81 = 0.06 | |
| | | | RRT 0.90 = 0.15 | |
| | | | RRT 0.93-0.94 = 0.1 | |
| | | | RRT 1.16-1.18 = 0.1 | |
| 50% PEG300 | 13.91 | 4.92 | RRT 0.70-0.71 = 0.06 | 0.06 |
| pH 5.0 at 40° C. | | | | |
| 50% PEG300 | 14.45 | 4.7 | RRT 0.61-.062 = 0.6 | 1.23 |
| pH 5.0 at 60° C. | | | RRT 0.70-0.71 = 0.22 | |
| | | | RRT 0.81 = 0.09 | |
| | | | RRT 0.90 = 0.13 | |
| | | | RRT 0.93-0.94 = 0.09 | |
| | | | RRT 1.16-1.18 = 0.09 | |

TABLE 5
Results at 2 weeks

| | Assay | | Impurities | Total |
|---|---|---|---|---|
| Sample | (mg/mL) | pH | present | Impurity |
| 50% PEG300 | 14.26 | 3.97 | RRT 0.66-0.67 = 0.05 | 0.25 |
| pH 4.0 at 40° C. | | | RRT 0.76 = 0.2 | |
| 50% PEG300 | 13.9 | 3.77 | RRT 0.66-0.67 = 0.42 | 2.01 |
| pH 4.0 at 60° C. | | | RRT 0.70-0.71 = 0.14 | |
| | | | RRT 0.76 = 0.73 | |
| | | | RRT 0.85 = 0.07 | |
| | | | RRT 0.93-0.94 = 0.44 | |
| | | | RRT 0.96 = 0.11 | |
| | | | RRT 1.16-1.18 = 0.1 | |
| 50% PEG300 | 14.51 | 5.02 | RRT 0.66-0.67 = 0.08 | 0.16 |
| pH 5.0 at 40° C. | | | RRT 0.76 = 0.08 | |
| 50% PEG300 | 14.34 | 4.69 | RRT 0.66-0.67 = 0.61 | 1.77 |
| pH 5.0 at 60° C. | | | RRT 0.76 = 0.37 | |
| | | | RRT 0.85 = 0.11 | |
| | | | RRT 0.93-0.94 = 0.31 | |
| | | | RRT 0.96 = 0.11 | |
| | | | RRT 0.97 = 0.12 | |
| | | | RRT 1.16-1.18 = 0.1 | |
| | | | RRT 1.22 = 0.05 | |

Another embodiment was prepared having a concentration of 25 mg/mL and the stability was tested over the course of 2 weeks at 40° C. and 60° C. A 25 mg/mL concentration was prepared using 50% polyethylene glycol at a pH of 4.0 and 5.0. The results of the stability study is presented in Table 6-8.

TABLE 6
Results at Initial Time Point

| | Assay | | Impurities | Total |
|---|---|---|---|---|
| Sample | (mg/mL) | pH | present | Impurity |
| 50% PEG300 | 22.97 | 4.01 | RRT 0.70-0.71 = 0.06 | 0.06 |
| pH 4.0 at 40° C. | | | | |
| 50% PEG300 | 23.87 | 4.86 | RRT 0.70-0.71 = 0.05 | 0.05 |
| pH 5.0 at 40° C. | | | | |

TABLE 7
Results at 1 week

| | Assay | | Impurities | Total |
|---|---|---|---|---|
| Sample | (mg/mL) | pH | present | Impurity |
| 50% PEG300 | 22.28 | 4.03 | RRT 0.70-0.71 = 0.13 | 0.13 |
| pH 4.0 at 40° C. | | | | |
| 50% PEG300 | 21.99 | 3.87 | RRT 0.61-.062 = 0.32 | 1.04 |
| pH 4.0 at 60° C. | | | RRT 0.70-0.71 = 0.36 | |
| | | | RRT 0.81 = 0.05 | |
| | | | RRT 0.90 = 0.14 | |
| | | | RRT 0.93-0.94 = 0.08 | |
| | | | RRT 1.16-1.18 = 0.09 | |
| 50% PEG300 | 22.91 | 4.88 | RRT 0.70-0.71 = 0.07 | 0.07 |
| pH 5.0 at 40° C. | | | | |
| 50% PEG300 | 22.76 | 4.64 | RRT 0.61-.062 = 0.66 | 1.48 |
| pH 5.0 at 60° C. | | | RRT 0.70-0.71 = 0.29 | |
| | | | RRT 0.81 = 0.1 | |
| | | | RRT 0.90 = 0.2 | |
| | | | RRT 0.93-0.94 = 0.11 | |
| | | | RRT 1.16-1.18 = 0.12 | |

TABLE 8
Results at 2 weeks

| | Assay | | Impurities | Total |
|---|---|---|---|---|
| Sample | (mg/mL) | pH | present | Impurity |
| 50% PEG300 | 23.17 | 4.05 | RRT 0.76 = 0.18 | 0.18 |
| pH 4.0 at 40° C. | | | | |
| 50% PEG300 | 22.39 | 4 | RRT 0.66-0.67 = 0.36 | 2.17 |
| pH 4.0 at 60° C. | | | RRT 0.70-0.71 = 0.11 | |
| | | | RRT 0.76 = 0.97 | |
| | | | RRT 0.85 = 0.06 | |
| | | | RRT 0.93-0.94 = 0.37 | |
| | | | RRT 0.97 = 0.12 | |
| | | | RRT 1.16-1.18 = 0.12 | |
| | | | RRT 1.22 = 0.07 | |
| 50% PEG300 | 21.75 | 4.72 | RRT 0.66-0.67 = 0.08 | 0.15 |
| pH 5.0 at 40° C. | | | RRT 0.81 = 0.07 | |
| 50% PEG300 | 23.37 | 4.65 | RRT 0.66-0.67 = 0.6 | 1.96 |
| pH 5.0 at 60° C. | | | RRT 0.70-0.71 = 0.05 | |
| | | | RRT 0.76 = 0.38 | |
| | | | RRT 0.85 = 0.11 | |
| | | | RRT 0.93-0.94 = 0.39 | |
| | | | RRT 0.96 = 0.06 | |
| | | | RRT 0.97 = 0.15 | |
| | | | RRT 1.16-1.18 = 0.15 | |
| | | | RRT 1.22 = 0.09 | |

Further, stability tests were conducted using different excipients with a 50 mg/mL concentration at a pH of 5.0. The results of the stability tests are presented in Tables 9-11 below.

TABLE 9
Results at Initial Time Point

| | Assay | | Impurities | Total |
|---|---|---|---|---|
| Sample | (mg/mL) | pH | present | Impurity |
| 50% PEG300 | 47.34 | 4.98 | RRT 0.70-0.71 = 0.07 | 0.07 |
| 10% EtOH | | | | |
| at 40° C. | | | | |
| 50% PEG300 | 41.17 | 4.86 | RRT 0.70-0.71 = 0.07 | 0.07 |
| 15% Glycerin | | | | |
| at 40° C. | | | | |
| 50% PEG300 | 47.03 | 4.86 | RRT 0.70-0.71 = 0.06 | 0.06 |
| 10% DMA | | | | |
| 50% PG | 45.33 | 4.86 | RRT 0.70-0.71 = 0.06 | 0.06 |
| 80% PG | 45.63 | 5.09 | RRT 0.70-0.71 = 0.06 | 0.06 |

TABLE 10
Results at 1 week

| | Assay | | Impurities | Total |
|---|---|---|---|---|
| Sample | (mg/mL) | pH | present | Impurity |
| 50% PEG300 | 48.17 | 4.54 | RRT 0.76 = 0.11 | 0.11 |
| 10% EtOH | | | | |
| at 40° C. | | | | |
| 50% PEG300 | 47.13 | 4.51 | RRT 0.66-0.67 = 0.22 | 0.88 |
| 10% EtOH | | | RRT 0.76 = 0.3 | |
| at 60° C. | | | RRT 0.93 = 0.07 | |
| | | | RRT 0.97 = 0.09 | |
| | | | RRT 1.18 = 0.1 | |
| | | | RRT 1.22 = 0.09 | |
| 50% PEG300 | 43.67 | 4.84 | RRT 0.76 = 0.09 | 0.09 |
| 15% Glycerin | | | | |
| at 40° C. | | | | |
| 50% PEG300 | 42.78 | 4.65 | RRT 0.66-0.67 = 0.32 | 1.26 |
| 15% Glycerin | | | RRT 0.76 = 0.33 | |
| at 60° C. | | | RRT 0.78 = 0.07 | |
| | | | RRT 0.85 = 0.07 | |
| | | | RRT 0.93 = 0.17 | |
| | | | RRT 0.97 = 0.1 | |
| | | | RRT 1.18 = 0.11 | |
| | | | RRT 1.22 = 0.16 | |
| 50% PEG300 | 48.42 | 4.77 | RRT 0.76 = 0.1 | .1 |
| 10% DMA | | | | |
| at 40° C. | | | | |
| 50% PEG300 | 46.9 | 4.82 | RRT 0.66-0.67 = 0.36 | 1.26 |
| 10% DMA | | | RRT 0.76 = 0.27 | |
| at 60° C. | | | RRT 0.85 = 0.07 | |
| | | | RRT 0.93 = 0.19 | |
| | | | RRT 0.97 = 0.1 | |
| | | | RRT 1.18 = 0.12 | |
| | | | RRT 1.22 = 0.15 | |
| 50% PG | 46.3 | 4.84 | RRT 0.76 = 0.07 | 0.07 |
| at 40° C. | | | | |
| 50% PG | 46.57 | 4.84 | RRT 0.76 = 0.21 | 0.28 |
| at 60° C. | | | RRT 0.89 = 0.07 | |
| 80% PG | 46.5 | 5.12 | RRT 0.76 = 0.07 | 0.07 |
| at 40° C. | | | | |
| 80% PG | 46.69 | 5.07 | RRT 0.76 = 0.07 | 0.25 |
| at 60° C. | | | RRT 0.89 = 0.12 | |
| | | | RRT 0.90 = 0.06 | |

TABLE 11
Results at 2 weeks

| | Assay | | Impurities | Total |
|---|---|---|---|---|
| Sample | (mg/mL) | pH | present | Impurity |
| 50% PEG300 | 48.27 | 4.41 | RRT 0.66-0.67 = <0.05 | 0.16 |
| 10% EtOH | | | RRT 0.76 = 0.16 | |
| at 40° C. | | | | |
| 50% PEG300 | 47.07 | 4.63 | RRT 0.66-0.67 = 0.24 | 1.7 |
| 10% EtOH | | | RRT 0.76 = 0.92 | |
| at 60° C. | | | RRT 0.78 = <0.05 | |
| | | | RRT 0.85 = 0.05 | |
| | | | RRT 0.89 = <0.05 | |
| | | | RRT 0.90 = <0.05 | |
| | | | RRT 0.93 = 0.15 | |
| | | | RRT 0.96 = <0.05 | |
| | | | RRT 0.97 = 0.12 | |
| | | | RRT 1.18 = 0.13 | |
| | | | RRT 1.22 = 0.1 | |
| 50% PEG300 | 44.14 | 4.74 | RRT 0.66-0.67 = 0.06 | 0.18 |
| 15% Glycerin | | | RRT 0.76 = 0.12 | |
| at 40° C. | | | | |
| 50% PEG300 | 41.77 | 4.69 | RRT 0.66-0.67 = 0.41 | 1.93 |
| 15% Glycerin | | | RRT 0.76 = 0.4 | |
| at 60° C. | | | RRT 0.78 = 0.13 | |
| | | | RRT 0.85 = 0.11 | |
| | | | RRT 0.89 = <0.05 | |
| | | | RRT 0.90 = <0.05 | |
| | | | RRT 0.93 = 0.33 | |
| | | | RRT 0.96 = 0.05 | |
| | | | RRT 0.97 = 0.17 | |
| | | | RRT 1.18 = 0.17 | |
| | | | RRT 1.22 = 0.15 | |
| 50% PEG300 | 48.46 | 4.68 | RRT 0.66-0.67 = 0.07 | 0.21 |
| 10% DMA | | | RRT 0.76 = 0.14 | |
| at 40° C. | | | | |
| 50% PEG300 | 47.08 | 4.88 | RRT 0.66-0.67 = 0.4 | 1.79 |
| 10% DMA | | | RRT 0.76 = 0.54 | |
| at 60° C. | | | RRT 0.78 = <0.05 | |
| | | | RRT 0.85 = 0.08 | |
| | | | RRT 0.89 = <0.05 | |
| | | | RRT 0.90 = <0.05 | |
| | | | RRT 0.93 = 0.27 | |
| | | | RRT 0.96 = 0.05 | |
| | | | RRT 0.97 = 0.15 | |
| | | | RRT 1.18 = 0.16 | |
| | | | RRT 1.22 = 0.15 | |
| 50% PG | 47.1 | 4.74 | RRT 0.66-0.67 = <0.05 | 0.09 |
| at 40° C. | | | RRT 0.76 = 0.09 | |
| 50% PG | 46.83 | 4.79 | RRT 0.66-0.67 = <0.05 | 0.65 |
| at 60° C. | | | RRT 0.76 = 0.42 | |
| | | | RRT 0.78 = <0.05 | |
| | | | RRT 0.85 = <0.05 | |
| | | | RRT 0.89 = 0.14 | |
| | | | RRT 0.90 = 0.08 | |
| | | | RRT 0.93 = <0.05 | |
| | | | RRT 0.96 = <0.05 | |
| | | | RRT 0.97 = <0.05 | |
| | | | RRT 1.18 = <0.05 | |
| | | | RRT 1.22 = <0.05 | |
| 80% PG | 46.91 | 5.06 | RRT 0.66-0.67 = <0.05 | 0.08 |
| at 40° C. | | | RRT 0.76 = 0.08 | |
| 80% PG | 47.2 | 5.05 | RRT 0.66-0.67 = <0.05 | 0.6 |
| at 60° C. | | | RRT 0.76 = 0.26 | |
| | | | RRT 0.78 = <0.05 | |
| | | | RRT 0.85 = 0.22 | |
| | | | RRT 0.89 = 0.12 | |
| | | | RRT 0.90 = <0.05 | |
| | | | RRT 0.93 = <0.05 | |
| | | | RRT 0.96 = <0.05 | |
| | | | RRT 0.97 = <0.05 | |
| | | | RRT 1.18 = <0.05 | |
| | | | RRT 1.22 = <0.05 | |

### Solubility of the Formulation as a Function of pH

A pH solubility study was performed to assess the feasibility of formulating a compound having Formula (I) as a simple buffered solution at a reasonable pH for a nasal formulation.

Methodology: A compound having Formula (I) and H2SO4 solution of concentration ˜107 mg/mL in 100 mM citric acid at pH 2.5 was prepared. About 150 μL aliquots of this solution were transferred into 6 separate glass HPLC vials. Each aliquot was titrated with 6N sodium hydroxide to different pH values in the range 3-5.5 as shown in Table 12. All the samples, including the solution at pH 2.5, were shaken overnight at room temperature using a gyratory shaker. After shaking, the samples were evaluated visually for the presence of precipitate and were then spin filtered through 0.45 μm syringe tip filters (4 mm diameter) at 3000 rpm for 20 min. The final pH values of the filtrates were measured. The filtrates were then diluted appropriately and analyzed for compound having Formula (I). H2SO4 concentration using the HPLC assay.

Results: The pH values of the samples prior to and after shaking overnight, appearance as well as concentrations of samples are shown in Table 12. Solutions prepared at target pH values of 2.5, 3.0 and 3.5 were clear (no excess solid present) and hence the concentration values for these samples do not represent saturation solubility values.

TABLE 12
pH-Solubility Results

| | | Final pH after | Appearance | Cmp. A.H₂SO₄ |
|---|---|---|---|---|
| Target | Initial | Shaking and | after | Concentration |
| pH | pH | Filtration | Shaking | (mg/mL) |
| 2.5 | 2.51 | 2.42 | Clear solution | 104.1 |
| 3.0 | 3.05 | 3.00 | Clear solution | 99.8 |
| 3.5 | 3.51 | 3.51 | Clear solution | 101.6 |
| 4.0 | 4.00 | 3.79 | Precipitate | 20.8 |
| 4.5 | 4.55 | 4.37 | Precipitate | 3.46 |
| 5.0 | 4.93 | 4.52 | Precipitate | 2.73 |
| 5.5 | 5.63 | 5.02 | Precipitate | 0.99 |

### Stability as a Function of pH

The compound having Formula (I) has been evaluated to have a pKa of 5.6 and a theoretical free base solubility of about 0.18 mg/mL. Thus, the inventors believe that the solubility of the drug becomes inadequate for the intended product concentration above about pH 3.6. Stability of the drug in solution could be an issue at such a low pH. The purpose of this experiment was to assess the stability of the drug as a function of pH.

A key consideration in such an experiment is the concentration of drug and buffer used. A buffer is used since the data is meaningless if the pH shifts dramatically during the experiment. However, it is desirable to keep the buffer at minimal strength to minimize the impact of buffer catalysis. Buffer strength can be minimized by keeping the drug concentration low. Keeping the drug concentration low also minimizes confounding the data with solubility limits (i.e. the drug precipitating out of solution).

Methodology: Buffer solutions containing ˜10 mM each of citric acid and glycine in the pH range 2-6 were prepared by titrating 250 mL of a single stock containing both buffer agents with 1N NaOH/1N HCl and removing 50 mL aliquots at predetermined pH values. Solutions at 1 mg/mL were prepared in buffers of pH 2-5 by dissolving ˜25 mg of Compounds having Formula (I). H2SO4 in ˜23 mL of a given buffer, adjusting the pH to the target and making up the volume to 25.0 mL in a volumetric flask. The solution at pH 6 was prepared similarly but at a lower drug concentration of 0.25 mg/mL using 6.68 mg of Compounds having Formula (I). H2SO4.

Each of the buffered solutions were filtered through a 0.2 μm polyether sulfone syringe filter into a volumetric flask, which had been previously rinsed with 70% ethanol and dried in a laminar flow hood. Following filtration, each buffered solution was split into 10 aliquots (9 aliquots of 2.5 mL each and 1 aliquot of 2 mL) in 5 mL serum vials previously rinsed with 70% ethanol and dried in a laminar flow hood. Filtration and preparation of aliquots were performed in a laminar flow hood. The 2 mL aliquot of each buffered solution was submitted for TO analysis (HPLC assay and related substances). Of the nine 2.5 mL aliquots, three aliquots were stored at each 40° C. and 60° C., two aliquots were stored at 25° C. and one aliquot was stored at −70° C. Samples stored at 40° C. and at 60° C. were evaluated for physical appearance, pH, assay and related substances after 1 week, 2 weeks and 4 weeks of storage. Samples at 25° C. and −70° C. served as controls to be analyzed if necessary.

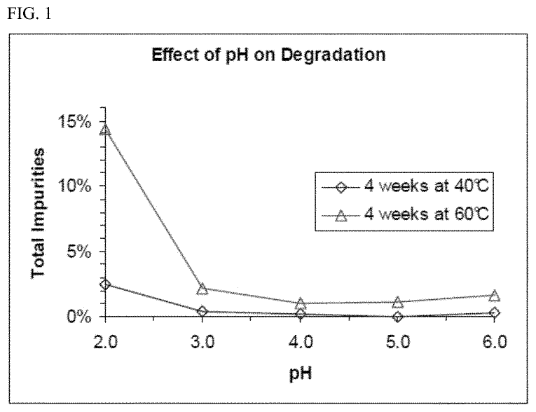

Results: The pH stability data are shown in Table 13. All samples remained clear, colorless and particle free for up to 4 weeks of storage at both 40° C. and at 60° C. The pH values for all the samples remained relatively constant for up to 4 weeks at both storage temperatures. Both the assay and impurities data indicate that the solution was significantly less stable at pH 2 as compared to the remaining pH values evaluated. No specific trends were observed in the pH range 3-6. The pH stability profiles at 60° C. are shown in FIG. 1 (% impurities).

TABLE 13
pH stability

| | Target | | 40° C. Storage | | | 60° C. Storage | | |
|---|---|---|---|---|---|---|---|---|
| Test | pH | initial | 1 wk | 2 wk | 4 wk | 1 wk | 2 wk | 4 wk |
| pH | 2 | 1.96 | 2.02 | 1.98 | 2.00 | 2.00 | 1.99 | 2.01 |
| | 3 | 3.02 | 3.05 | 3.05 | 3.06 | 3.07 | 3.03 | 3.07 |
| | 4 | 4.00 | 3.94 | 3.96 | 3.96 | 3.94 | 3.96 | 3.97 |
| | 5 | 5.03 | 4.90 | 4.95 | 4.97 | 4.92 | 4.93 | 4.98 |
| | 6 | 6.05 | 5.88 | 6.02 | 5.98 | 5.87 | 6.03 | 6.01 |
| Assay | 2 | 1.02 | 0.997 | 0.975 | 0.970 | 0.929 | 0.754 | 0.754 |
| | 3 | 0.984 | 0.984 | 0.987 | 0.991 | 0.971 | 0.952 | 0.952 |
| | 4 | 1.01 | 0.999 | 1.006 | 1.012 | 0.992 | 0.979 | 0.979 |
| | 5 | 1.01 | 0.989 | 0.992 | 0.977 | 0.977 | 0.947 | 0.947 |
| | 6 | 0.260 | 0.257 | 0.253 | 0.251 | 0.247 | 0.234 | 0.234 |
| Impurities | 2 | n.d. | 0.58 | 1.48 | 2.49 | 3.83 | 9.89 | 14.34 |
| | 3 | n.d. | n.d. | 0.19 | 0.41 | 0.71 | 1.20 | 2.19 |
| | 4 | n.d. | n.d. | 0.17 | 0.22 | n.d. | 0.52 | 1.02 |
| | 5 | n.d. | n.d. | 0.26 | n.d | n.d. | 0.62 | 1.10 |
| | 6 | n.d. | n.d. | 0.25 | 0.28 | 0.56 | 0.87 | 1.65 |

n.d. = not detected

Stability as a Function of pH—Narrow Range of pH

The pH stability study described above showed that the compound was unstable at pH 2.0 but more stable at higher pH values. Accordingly, the stability of the compound was evaluated in the pH range 2-3.6.

Methodology: Buffer solutions containing ˜10 mM each of citric acid and glycine in the pH range 2-3.6 were prepared by titrating 250 mL of a single stock containing both buffer agents with 1N NaOH/1N HCl and removing 50 mL aliquots at predetermined pH values. Solutions at 1 mg/mL were prepared in the buffers by dissolving ˜25 mg of compound in ˜23 mL of a given buffer, adjusting the pH to the target and making up the volume to 25.0 mL in a volumetric flask.

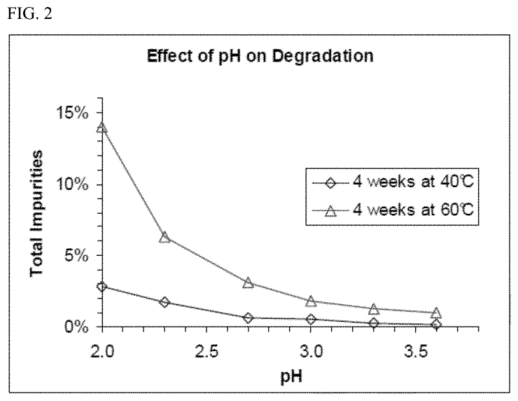

Each of the buffered solutions was filtered through a 0.2 m polyether sulfone syringe filter into a volumetric flask previously rinsed with 70% ethanol and dried in a laminar flow hood. Following filtration, each buffered solution was split into 9 aliquots in 5 mL serum vials previously rinsed with 70% ethanol and dried in a laminar flow hood. Filtration and preparation of aliquots were performed in a laminar flow hood. Of the nine aliquots, three aliquots were stored at each 40° C. and 60° C., two aliquots were stored at 25° C. and one aliquot was submitted for T0 testing. Samples were evaluated for pH, assay and related substances at TO and, following 1 week, 2 weeks and 4 weeks of storage at 40° C. and 60° C. Samples stored at 25° C. served as controls to be analyzed if necessary. The results are presented in Table 14 and FIG. 2.

The most notable result from this study is that during both 40° C. and 60° C. storage, impurity levels decrease significantly with increasing pH across the full pH range studied. This means that any increase in the pH of the formulation leads to an improvement in stability within the pH range in which an aqueous formulation is feasible.

TABLE 14
pH Stability of Cmp. A - Narrow pH Range

| | Target | | 40° C. Storage | | | 60° C. Storage | | |
|---|---|---|---|---|---|---|---|---|
| Test | pH | initial | 1 wk | 2 wk | 4 wk | 1 wk | 2 wk | 4 wk |
| pH | 2.0 | 2.00 | 2.00 | 1.98 | 1.98 | 2.00 | 1.97 | 1.89 |
| | 2.3 | 2.33 | 2.36 | 2.32 | 2.33 | 2.36 | 2.34 | 2.28 |
| | 2.7 | 2.72 | 2.73 | 2.72 | 2.73 | 2.75 | 2.74 | 2.67 |
| | 3.0 | 3.02 | 3.04 | 3.05 | 3.04 | 3.05 | 3.04 | 3.00 |
| | 3.3 | 3.31 | 3.35 | 3.35 | 3.34 | 3.36 | 3.34 | 3.34 |
| | 3.6 | 3.62 | 3.64 | 3.70 | 3.64 | 3.65 | 3.70 | 3.64 |
| Assay | 2.0 | 0.981 | 0.955 | 0.959 | 0956 | 0.913 | 0.851 | 0.786 |
| | 2.3 | 0.987 | 0.984 | 0.993 | 0.995 | 0.961 | 0.932 | 0.916 |
| | 2.7 | 0.987 | 0.981 | 0.985 | 1.019 | 0.978 | 1.144 | 0.987 |
| | 3.0 | 1.005 | 1.01 | 0.987 | 1.027 | 0.991 | 0.975 | 0.987 |
| | 3.3 | 1.019 | 1.02 | 1.01 | 1.047 | 0.97 | 0.995 | 1.027 |
| | 3.6 | 0.996 | 0.991 | 0.985 | 0.991 | 0.979 | 0.958 | 0.979 |
| Impurities | 2.0 | 0.14 | 0.99 | 1.55 | 2.84 | 3.87 | 7.56 | 13.99 |
| | 2.3 | 0.09 | 0.47 | 0.63 | 1.70 | 1.81 | 3.53 | 6.30 |
| | 2.7 | n.d. | 0.22 | 0.37 | 0.61 | 0.97 | 1.76 | 3.9 |
| | 3.0 | n.d. | 0.21 | 0.34 | 0.58 | 0.61 | 1.27 | 1.82 |
| | 3.3 | n.d. | n.d. | n.d. | 0.25 | 0.41 | 0.69 | 1.26 |
| | 3.6 | 0.12 | n.d. | n.d. | 0.17 | 0.36 | 0.62 | 0.99 |

n.d. = not detected

### Drug Substance Stability

Methodology: Approximately 100 mg of the API (Lot 009M9B058) were stored at each of 40° C. and 60° C. in closed Type I glass vials. Samples were analyzed for appearance and percent purity at T0 and after 1 week, 2 weeks and 4 weeks of storage.

Results: As shown in Table 15, the API was quite stable for 4 weeks even at 60° C. Visually, there were no signs of degradation (i.e. discoloration etc.).

TABLE 15
Drug Substance Stability

| | Storage | T = | T = | T = | T = |
|---|---|---|---|---|---|
| Test | Condition | 0 | 1 week | 2 week | 4 week |
| Appearance | 40° C. | White | White | White | White |
| | 60° C. | powder | powder | powder | powder |
| Purity (%) | 40° C. | 100.00 | 99.92 | 99.90 | 100.00 |
| | 60° C. | | 99.76 | 100.00 | 100.00 |
| Related | 40° C. | n.d. | Total 0.08 | Total 0.10 | n.d. |
| Substances | 60° C. | | Total 0.24 | n.d. | n.d. |

n.d. = not detected

### Simple Prototype Stability

An experiment was conducted to determine the stability of the API in simple formulation prototypes. The solutions were prepared having a concentration of 25 mg/mL of API as free base which corresponds to 35 mg/mL of compound having Formula (I). H2SO4.

Methodology: A total of 9 prototypes were prepared that included a simple buffered solution, co-solvent systems as well as non-aqueous solutions as shown in Table 16.

TABLE 16
Cmp. A Prototypes for Stability Evaluation

| Prototype # | Description |
|---|---|
| 1 | 35 mg/mL compound having Formula (I). |
| | H2SO4 in 50 mM citrate, pH 3.0 |
| 2 | 35 mg/mL compound having Formula (I). |
| | H2SO4 in 50% v/v PEG-400 in water |
| 3 | 35 mg/mL compound having Formula (I). |
| | H2SO4 in 50% v/v PEG-400 in water |
| | adjusted to pH 4.4 with 1N NaOH |
| 4 | 30 mg/mL compound having Formula (I). |
| | H2SO4 in 100% PEG-400 |
| 5 | 35 mg/mL compound having Formula (I). |
| | H2SO4 in 100% propylene glycol |
| 6 | 35 mg/mL compound having Formula (I). |
| | H2SO4 in 50% v/v PEG-400 in acetate solution |
| | (~40 mM sodium acetate and ~100 |
| | mM sodium hydroxide) |
| 7 | 35 mg/mL compound having Formula (I). |
| | H2SO4 in 50% v/v propylene glycol in |
| | acetate solution (~40 mM sodium |
| | acetate and ~100 mM sodium hydroxide) |
| 8 | 35 mg/mL compound having Formula (I). |
| | H2SO4v/v PEG-400 in acetate solution |
| | (~40 mM ammonium acetate and ~100 |
| | mM ammonium hydroxide) |
| 9 | 35 mg/mL compound having Formula (I). |
| | H2SO4 in 70% v/v propylene glycol in acetate |
| | solution (~40 mM ammonium acetate |
| | and ~100 mM ammonium hydroxide) |

Prototypes 6, 7 and 8 resulted in significant precipitation during preparation and hence were not evaluated further. The remaining prototypes were stored at 2-8° C., RT, 40° C. and 60° C. in serum vials. Samples stored at 2-8° C. and RT were analyzed at selected timepoints.

Results: Data is provided in Table 17 through Table 22. The aqueous buffered formulation (Prototype #1) was found to be stable at room temperature and under refrigeration, but some degradation was observed under accelerated conditions. A very small amount of precipitate, described as one or two snowflake-like particles, has been observed after 2 weeks at 60° C. and after 8 weeks at 40° C. Since the appearance of this precipitate during 60° C. storage coincided with an actual decrease in the primary impurity peak, the possibility of the degradant being insoluble is under investigation.

Prototype #2 was unstable in, 50% PEG/500% water without pH adjustment. The instability was presumably due to the low pH of this formulation. The drug was found to be much more stable in Prototype #3, which has the same 5000 PEG base but also contains sufficient sodium hydroxide to bring the apparent pH up to about 4.5. Similarly, good stability was seen in Prototype #9, 70% propylene glycol/30% water with ammonium acetate buffer to bring the apparent pH to about 5.3. Good stability was also seen in simple solutions of the salt in PEG or propylene glycol, presumably due to the absence of water as a reactant.

TABLE 17
Stability of Prototype #1
Formulation #1

| | | | | 1 | 2 | 4 |
|---|---|---|---|---|---|---|
| Parameter | Temperature | | Initial | week | weeks | weeks |
| pH | 2-8° | C. | 2.97 | 3.04 | — | — |
| | RT | | | 3.04 | — | 3.07 |
| | 40° | C. | | 2.99 | 3.01 | 3.06 |
| | 60° | C. | | 3.01 | 3.02 | 2.97 |
| Assay | 2-8° | C. | 35.73 | 36.22 | NA | NA |
| | RT | | | 35.93 | — | 35.31 |
| | 40° | C. | | 34.83 | 35.40 | 33.96 |
| | 60° | C. | | 37.90 | 34.63 | 31.89 |
| Impurities | 2-8° | C. | n.d. | n.d. | — | — |
| | RT | | | n.d. | — | n.d. |
| | 40° | C. | | n.d. | 0.35 | 0.52 |
| | 60° | C. | | 0.73 | 0.49 | 0.51 |

TABLE 18
Stability of Prototype #2
Formulation #2

| | | | 1 | 2 | 4 |
|---|---|---|---|---|---|
| Parameter | Temperature | Initial | week | weeks | weeks |
| pH | RT | 2.00 | 2.01 | NA | 2.07 |
| | 40° C. | | 2.04 | 2.03 | 2.11 |
| | 60° C. | | 2.04 | 2.14 | 2.36 |
| Assay | RT | 33.93 | 35.26 | NA | 34.47 |
| | 40° C. | | 35.30 | 34.81 | 33.92 |
| | 60° C. | | 33.70 | 31.77 | 27.45 |
| Impurities | RT | n.d. | 0.39 | 0.39 | 1.10 |
| | 40° C. | | 0.92 | 0.92 | 2.53 |
| | 60° C. | | 2.11 | 2.11 | 11.99 |

TABLE 19
Stability of Cmp. A Formulation Prototype #3
Formulation #3

| | | | | 1 | 2 | 4 |
|---|---|---|---|---|---|---|
| Parameter | Temperature | | Initial | week | weeks | weeks |
| pH | 2-8° | C. | 4.38 | 4.51 | NA | NA |
| | RT | | | 4.40 | NA | 4.52 |
| | 40° | C. | | 4.20 | 4.43 | 4.41 |
| | 60° | C. | | 4.16-3.88 | 4.22 | 4.27 |
| Assay | 2-8° | C. | 22.17 | 37.31 | NA | NA |
| | RT | | | 36.23 | NA | 36.03 |
| | 40° | C. | | 35.66 | 36.53 | 35.96 |
| | 60° | C. | | 35.33 | 34.70 | 34.14 |
| Impurities | 2-8° | C. | n.d. | n.d. | NA | NA |
| | RT | | | n.d. | NA | n.d. |
| | 40° | C. | | n.d. | n.d. | 0.90 |
| | 60° | C. | | 0.62 | 1.50 | 1.91 |

TABLE 20
Stability of Prototype #4
Formulation #4

| | | | | 1 | 2 | 4 |
|---|---|---|---|---|---|---|
| Parameter | Temperature | | Initial | week | weeks | weeks |
| Assay | 2-8° | C. | 27.94 | 30.18 | NA | NA |
| | RT | | | 30.20 | NA | 30.31 |
| | 40° | C. | | 29.91 | 29.89 | 30.19 |
| | 60° | C. | | 29.24 | 29.60 | 29.97 |
| Impurities | 2-8° | C. | n.d. | n.d. | NA | NA |
| | RT | | | n.d. | NA | n.d. |
| | 40° | C. | | n.d. | n.d. | n.d. |
| | 60° | C. | | 0.50 | 0.50 | 0.37 |

TABLE 21
Stability of Prototype #5
Formulation #5:

| | | | | 1 | 2 | 4 |
|---|---|---|---|---|---|---|
| Parameter | Temperature | | Initial | week | weeks | weeks |
| Assay | 2-8° | C. | 28.97 | 36.22 | NA | NA |
| | RT | | | 36.23 | NA | 36.00 |
| | 40° | C. | | 35.16 | 35.63 | 36.03 |
| | 60° | C. | | 35.56 | 35.33 | 35.56 |
| Impurities | 2-8° | C. | n.d. | n.d. | NA | NA |
| | RT | | | n.d. | NA | n.d. |
| | 40° | C. | | n.d. | 0.12 | 0.27 |
| | 60° | C. | | 0.23 | 0.74 | 0.99 |

TABLE 22
Stability of Prototype #9
Formulation #9

| | | | | 1 | 2 | 4 |
|---|---|---|---|---|---|---|
| Parameter | Temperature | | Initial | week | weeks | weeks |
| pH | 2-8° | C. | 5.28 | 5.27 | NA | |
| | RT | | | NA | NA | |
| | 40° | C. | | 5.26 | 5.35 | |
| | 60° | C. | | 5.25 | 5.34 | |
| Assay | 2-8° | C. | 35.50 | 34.40 | NA | |
| | RT | | | NA | NA | |
| | 40° | C. | | 34.36 | 34.62 | |
| | 60° | C. | | 34.69 | 34.55 | |
| Impurities | 2-8° | C. | n.d. | n.d. | NA | |
| | RT | | | NA | NA | |
| | 40° | C. | | n.d. | n.d. | |
| | 60° | C. | | n.d. | 0.39 | |

The formulations as disclosed above are placed in a suitable nasal dropper, nasal spray, atomizer, nebulizer or other device suitable for administration to the nasal cavity.

From the narrow range pH stability study, it is clear that even in the upper part of the range studied, stability is substantially improved as the pH increases. The upper limit of the pH is defined by drug solubility, so that a decrease in the concentration of drug that needs to be dissolved will allow the product to be formulated at a higher pH, which will improve the stability of the product. Specifically, reducing the concentration by half allows the formulation pH to be increased by about 0.3 units, and as can be seen in Table 6, a 0.3 unit pH increase substantially improves stability of the formulation. Based on this consideration, it was decided to reduce the Cmp. A concentration in the formulation from 25 mg/mL to 10 mg/mL and to increase the target pH from 3.0 to 3.2. The buffer concentration was proportionately decreased from 50 mM to 20 mM. The final formulation is provided in Table 23.

TABLE 23
Final Formulation

| Component | Function | Concentration | % w/w |
|---|---|---|---|
| Compound having | Active | 10 mg/mL | 1.386 |
| Formula (I) | | (as free base) | (as salt) |
| Citric acid, | Buffer | 20 mM | 0.384 |
| Anhydrous, USP | | | |
| 1N Sodium Hydroxide | adjust pH to 3.2 | q.s | q.s. |
| Sterile Water for | solvent | q.s. | q.s. |
| Injection, USP | | | |

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “of”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A nasal formulation comprising from about 0.01% w/w to about 10% w/w of a compound of Formula (I) and a pharmaceutically acceptable excipient, wherein the compound of Formula (I) is selected from: 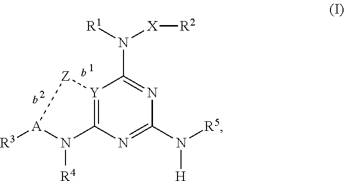
wherein:
R¹ and R² are independently H, alkyl, substituted alkyl, cycloalkyl, substituted cycloalkyl, alkenyl, substituted alkenyl, alkynyl, substituted alkynyl, phenyl, substituted phenyl, phenylalkyl, substituted phenylalkyl, aryl, substituted aryl, arylalkyl, substituted arylalkyl, heteroarylalkyl, substituted heteroarylalkyl, heteroaryl or substituted heteroaryl; or R¹ and R² combine as to form a biradical selected from the group consisting of 3-hydroxy-pentane-1,5-diyl, 6-hydroxy-cycloheptane-1,4-diyl, propane-1,3-diyl, butane-1,4-diyl and pentane-1,5-diyl;
R³ is H, alkyl, substituted alkyl, alkynyl, substituted alkynyl, cycloalkyl, substituted cycloalkyl, alkenyl, substituted alkenyl, —NR¹R², —C(O)OR¹, acyl, or aryl;
R⁴ is H, alkyl, or substituted alkyl;
R⁵ is H, alkyl, propargylic, substituted propargylic, homopropargylic, substituted homopropargylic, substituted alkyl, cycloalkyl, substituted cycloalkyl, alkenyl, substituted alkenyl, —OR¹, —NR¹R², —C(O)OR¹, acyl, aryl, substituted aryl, heteroaryl, substituted heteroaryl, heterocyclic, or substituted heterocyclic; or R³ and R⁵ combine as to form a biradical selected from the group consisting of 3,6,9-trioxa-undecane-1,11-diyl and 3,6-dioxa-octane-1,8-diyl;
R⁶ is H, alkyl, substituted alkyl or alkenyl;
X is a bond, O or NR⁴; and,
Y is N, CR⁶ or C; wherein:
The compound Y is N or CR⁶, then bond b¹ is nil and: (i) Z is H, bond b² is a single bond, and A is CH; or, (ii) Z is nil, bond b² is nil, and A is a single bond; and,
if Y is C, then bond b¹ is a single bond, and: (i) Z is CH₂, bond b² is a single bond, and A is CH; or, (ii) Z is CH, bond b² is a double bond, and A is C;
or a salt thereof; wherein the formulation is contained in a nasal delivery device.

2. The nasal formulation of claim 1, wherein pharmaceutically acceptable excipient comprises a liquid.

3. The nasal formulation of claim 1, wherein the compound of Formula (I) is dissolved in the liquid.

4. The nasal formulation of claim 1, wherein the compound of Formula (I) is suspended in the liquid.

5. The nasal formulation of claim 1, wherein the pharmaceutically acceptable excipient is a collection of particles.

6. The nasal formulation of claim 1, wherein the compound of Formula (I) is dispersed within each particle.

7. The nasal formulation of claim 1, wherein the compound of Formula (I) is coated on each particle.

8. The nasal formulation of claim 1, wherein the pharmaceutically acceptable excipient is a cream, ointment or gel.

9. The nasal formulation of claim 1, wherein the nasal formulation of maintains at least 90% of the compound after accelerated storage conditions of 40° C. at 75% relative humidity for 2 weeks.

10. The nasal formulation of claim 1, wherein the pH of the formulation is from about 3.5 to about 5.5.

11. The nasal formulation of claim 1, wherein the pH is from about 4 to about 5.

12. The nasal formulation of claim 1, wherein the pH is selected from about 4.0, about 4.5 or about 5.0.

13. The nasal formulation of claim 1, wherein the concentration of the compound is from about 1 mg/mL to about 100 mg/mL.

14. The nasal formulation of claim 1, wherein the excipient is selected from water, ethanol, polyalkylene glycol, alkylene glycol, cyclodextrin, saline, ringers solution, dextrose, polyethylene glycol-hydroxystearate or a combination thereof.

15. The nasal formulation of claim 14, wherein the excipient comprises ethanol.

16. The nasal formulation of claim 15, wherein the excipient comprises ethanol in an amount of from about 1% to about 30%.

17. The nasal formulation of claim 14, wherein the excipient comprises propylene glycol.

18. The nasal formulation of claim 17, wherein the excipient comprises propylene glycol in an amount of from about 20% to about 100%.

19. The nasal formulation of claim 14, wherein the excipient comprises polyethylene glycol.

20. The nasal formulation of claim 19, wherein the excipient comprises polyethylene glycol in an amount of from about 20% to about 100%.

21. The nasal formulation of claim 14, wherein the excipient comprises hydroxypropyl-β-cyclodextrin.

22. The nasal formulation of claim 21, wherein the excipient comprises hydroxypropyl-β-cyclodextrin in an amount of from about 1% to about 50%.

23. The nasal formulation of claim 1, further comprising a buffer solution.

24. The nasal formulation of claim 23, wherein the buffer solution comprises a citric acid, glycine, citrate or acetate.

25. The nasal formulation of claim 1, wherein the compound is N-(4,6-Bis-n-propylamino-[1,3,5]triazin-2-yl)-O,N-dimethyl-hydroxylamine or a pharmaceutically acceptable salt thereof.

26. The nasal formulation of claim 1, wherein the compound is the hydrogen sulfate salt of N-(4,6-Bis-n-propylamino-[1,3,5]triazin-2-yl)-O,N-dimethyl-hydroxylamine.

27. A method of providing respiratory stimulation comprising administering intranasally a nasal formulation of claim 1.

28. A method of treating respiratory depression comprising nasally administering a formulation of claim 1.

29. The method of claim 28, wherein the respiratory depression is caused by an opioid agent.

30. The method of claim 28, wherein the respiratory depression is caused by a non-opioid agent.

31. The method of claim 28, wherein the respiratory depression is caused by inflammation.

32. The method of claim 28, wherein the respiratory depression is caused by infection.
